(19) United States
(12) Patent Application Publication
(10) Pub. No.: US 2024/0124455 A1
(43) Pub. Date: Apr. 18, 2024

(54) TRICYCLIC COMPOUNDS AS ANTICANCER AGENTS

(71) Applicant: JINGRUI BIOPHARMA CO., LTD., Suzhou, Jiangsu (CN)

(72) Inventors: Ying HAN, Suzhou, Jiangsu (CN); Dapeng LI, Suzhou, Jiangsu (CN); Huajun LONG, Suzhou, Jiangsu (CN); Tong WANG, Suzhou, Jiangsu (CN); Zhiyu YIN, Suzhou, Jiangsu (CN); Yu WANG, Suzhou, Jiangsu (CN)

(21) Appl. No.: 18/262,448

(22) PCT Filed: Jan. 21, 2022
(86) PCT No.: PCT/CN2022/073097

§ 371 Date: Jul. 21, 2023

(30) Foreign Application Priority Data
Jan. 22, 2021 (WO) PCT/CN2021/073348

(51) Int. Cl. C07D 471/14; A61P 35/00

(52) U.S. Cl. CPC C07D 471/14; A61P 35/00

(57) ABSTRACT

The present invention is directed to tricyclic compounds, pharmaceutically acceptable compositions comprising compounds of the invention and methods of using said compositions in the treatment of various disorders.

### FIELD OF THE INVENTION

The present invention is directed to novel tricyclic compounds (formula I, I-1 and I-2) which are bromodomain and extra-terminal (BET) inhibitors, their synthesis and their use for treating diseases.

### BACKGROUND OF THE INVENTION

The genomes of eukaryotic organisms are highly organized within the nucleus of the cell. The long strands of duplex DNA are wrapped around an octamer of histone proteins to form a nucleosome. This basic unit is then further compressed by the aggregation and folding of nucleosomes to form a highly condensed chromatin structure. A range of different states of condensation are possible, and the tightness of this structure varies during the cell cycle, being most compact during the process of cell division. There has been appreciation recently that chromatin templates form a fundamentally important set of gene control mechanisms referred to as epigenetic regulation. By conferring a wide range of specific chemical modifications to histones and DNA (such as acetylation, methylation, phosphorylation, ubiquitinylation and SUMOylation) epigenetic regulators modulate the structure, function and accessibility of our genome, thereby exerting a huge impact in gene expression.

Histone acetylation is most usually associated with the activation of gene transcription, as the modification loosens the interaction of the DNA and the histone octomer by changing the electrostatics. In addition to this physical change, specific proteins bind to acetylated lysine residues within histones to read the epigenetic code. Bromodomains are small (˜110 amino acid) distinct domains within proteins that bind to acetylated lysine residues commonly but not exclusively in the context of histones. There is a family of around 50 proteins known to contain bromodomains, and they have a range of functions within the cell. The BET family of bromodomain containing proteins comprises 4 proteins (BRD2, BRD3, BRD4 and BRD-T) which contain tandem bromodomains capable of binding to two acetylated lysine residues in close proximity, increasing the specificity of the interaction.

BRD2 and BRD3 are reported to associate with histones along actively transcribed genes and may be involved in facilitating transcriptional elongation (Leroy et al., Mol. Cell. 2008 30(1):51-60), while BRD4 appears to be involved in the recruitment of the pTEF-I3 complex to inducible genes, resulting in phosphorylation of RNA polymerase and increased transcriptional output (Hargreaves et al., Cell, 2009 138(1): 1294145).

All family members have been reported to have some function in controlling or executing aspects of the cell cycle, and have been shown to remain in complex with chromosomes during cell division—suggesting a role in the maintenance of epigenetic memory. In addition some viruses make use of these proteins to tether their genomes to the host cell chromatin, as part of the process of viral replication (You et al., Cell, 2004 117(3):349-60).

Accordingly, there is a need for compounds that modulate the activity of the BET family of proteins, such as BRD4, that can be used to treat BET protein-associated diseases such as cancer. The compounds of the invention help meet this need.

### SUMMARY OF THE INVENTION

In one aspect, there is provided a compound of formula I, a pharmaceutically acceptable salt thereof or stereoisomer thereof:

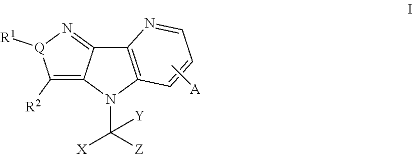

wherein constituent members are defined herein.

In another aspect, there is provided a pharmaceutical composition comprising a compound of the invention or a pharmaceutically acceptable salt thereof or stereoisomer thereof and one or more pharmaceutically acceptable carriers, diluents or excipients.

In another aspect, there is provided the use of a compound of the invention, a pharmaceutically acceptable salt thereof or stereoisomer thereof, in the manufacture of a medicament for the treatment of diseases or conditions for which a bromodomain inhibitor is indicated.

In yet another aspect, there is provided a method for the treatment of diseases or conditions for which a bromodomain inhibitor is indicated, comprising administering to a subject in need a compound of the invention, a pharmaceutically acceptable salt thereof or stereoisomer thereof.

### DETAILED DESCRIPTION OF THE INVENTION

In the first aspect of the present invention, the present application provides a compound of the formula I, a pharmaceutically acceptable salt thereof or stereoisomer thereof:

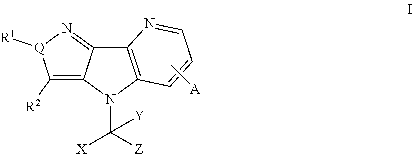

wherein:
Q is selected from N, O and S, provided that when Q is O or S, R¹ is absent;
A is selected from the following:

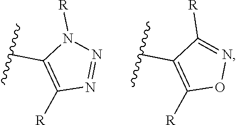

each of R is independently selected from hydrogen, optionally substituted (C₁-C₆) alkyl, halogen, and —CD₃;
X and Y are independently selected from phenyl; 6-membered heteroaryl containing 1 or 2 heteroatoms selected from N; 6-membered heterocyclic containing 1 or 2 heteroatoms selected from O, S; or 6-membered carbocyclic; and each of which at each occurrence is independently optionally substituted with hydrogen, —C₁₋₃ alkyl or halogen;
Z is selected from hydrogen, —F, —Cl, —OH, —C₁₋₃ alkyl or —C₁₋₃ alkoxy;
R¹ is selected from halogen, optionally substituted (C₁-C₆) alkyl, optionally substituted (C₂-C₆) alkenyl, optionally substituted (C₂-C₆) alkynyl;
R² is selected from —COOR²¹, and —(CH₂)ₙ—CR²²R²³—OH, wherein R²¹ is hydrogen, or optionally substituted (C₁-C₆) alkyl, each of R²² and R²³ is selected from hydrogen, halogen, and —C₁₋₆ alkyl; n is selected from 0, 1, 2, 3, 4, 5 or 6.

In some embodiments, the compound is of formula I-1:

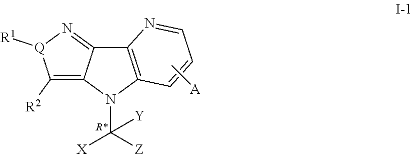

R* in the formula I-1 indicates that the absolute configuration of the carbon that contacts with the X, Y and Z is R configuration when the carbon is chiral carbon.

In some embodiments, the compound is of formula I-2:

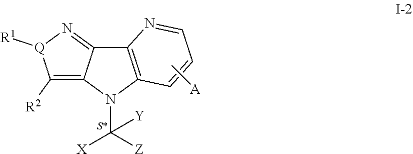

S* in the formula I-2 indicates that the absolute configuration of the carbon that contacts with the X, Y and Z is S configuration when the carbon is chiral carbon.

In one embodiment of the compound of the invention, a pharmaceutically acceptable salt thereof or stereoisomer thereof, Q is N. In one embodiment of the compound of the invention, a pharmaceutically acceptable salt thereof or stereoisomer thereof, Q is S. In one embodiment of the compound of the invention, a pharmaceutically acceptable salt thereof or stereoisomer thereof, Q is O.

In one embodiment of the compound of the invention, a pharmaceutically acceptable salt thereof or stereoisomer thereof, R¹ is selected from C₁₋₆ alkyl; wherein one or more hydrogen atoms on the C₁₋₆ alkyl group are optionally substituted by deuterium. In one embodiment of the compound of the invention, a pharmaceutically acceptable salt thereof or stereoisomer thereof, R¹ is selected from methyl, ethyl, propyl, or isopropyl. In one embodiment of the compound of the invention, a pharmaceutically acceptable salt thereof or stereoisomer thereof, R¹ is selected from —CH₃ or —CD₃.

In one embodiment of the compound of the invention, a pharmaceutically acceptable salt thereof or stereoisomer thereof, R² is selected from —COOR²¹, and —(CH₂)ₙ—CR²²R²³—OH, wherein R²¹ is (C₁-C₆) alkyl, each of R²² and R²³ is selected from —C₁₋₆ alkyl; n is selected from 0, 1, 2, 3, 4, 5 or 6. In one embodiment of the compound of the invention, a pharmaceutically acceptable salt thereof or stereoisomer thereof, R² is selected from —COOR²¹, and —(CH₂)ₙ—CR²²R²³—OH, wherein R²¹ is methyl, ethyl, propyl, or isopropyl, each of R²² and R²³ is selected from methyl, ethyl, propyl, or isopropyl, n is selected from 0, 1, 2, 3, 4, 5 or 6. In one embodiment of the compound of the invention, a pharmaceutically acceptable salt thereof or stereoisomer thereof, R₂ is selected from —COOR²¹, and —(CH₂)ₙ—CR²²R²³—OH, wherein R₂₁ is —CH₃, each of R₂₂ and R₂₃ is —CH₃; n is selected from 0. In one embodiment of the compound of the invention, a pharmaceutically acceptable salt thereof or stereoisomer thereof, R₂ is —C(CH₃)₂—OH.

In one embodiment of the compound of the invention, a pharmaceutically acceptable salt thereof or stereoisomer thereof, A is selected from the following:

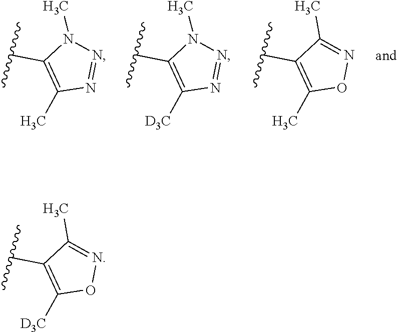

In one embodiment of the compound of the invention, a pharmaceutically acceptable salt thereof or stereoisomer thereof, A is selected from the following:

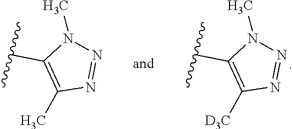

In one embodiment of the compound of the invention, a pharmaceutically acceptable salt thereof or stereoisomer thereof, X and Y are independently selected from phenyl, 6-membered heteroaryl containing 1 or 2 heteroatoms selected from N; 6-membered saturated heterocyclic containing 1 or 2 heteroatoms selected from O, S; or 6-membered saturated carbocyclic, each of which at each occurrence is independently optionally substituted with —C₁₋₃ alkyl or halogen. In one embodiment of the compound of the invention, a pharmaceutically acceptable salt thereof or stereoisomer thereof, X and Y are independently selected from phenyl, 6-membered heteroaryl containing 1 heteroatom selected from N; 6-membered saturated heterocyclic containing 1 heteroatom selected from O, S; or 6-membered saturated carbocyclic, each of which at each occurrence is independently optionally substituted with —C₁₋₃ alkyl or halogen. In one embodiment of the compound of the invention, a pharmaceutically acceptable salt thereof or stereoisomer thereof, X and Y are independently selected from phenyl, 6-membered heteroaryl containing 1 heteroatom selected from N; 6-membered saturated heterocyclic containing 1 heteroatom selected from O; or 6-membered saturated carbocyclic, each of which at each occurrence is independently optionally substituted with —CH₃, —F or —Cl. In one embodiment of the compound of the invention, a pharmaceutically acceptable salt thereof or stereoisomer thereof, X and Y are independently selected from phenyl;

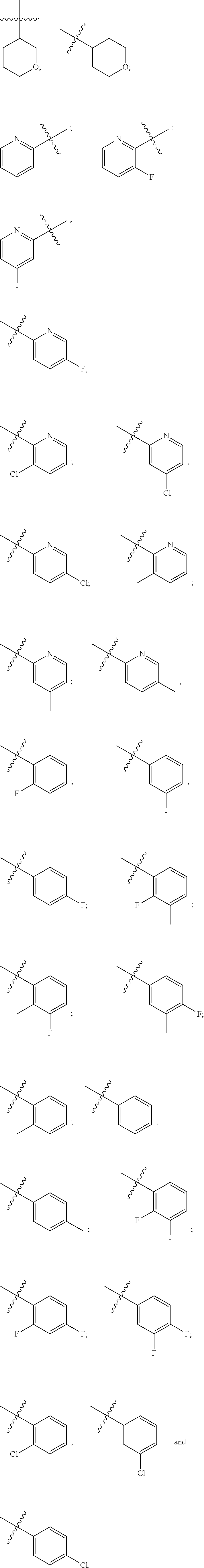

In one embodiment of the compound of the invention, a pharmaceutically acceptable salt thereof or stereoisomer thereof, X and Y are independently selected from phenyl;

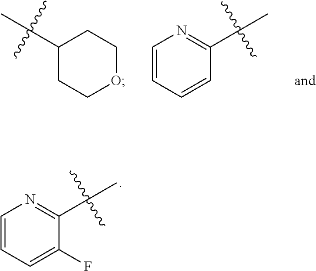

In one embodiment of the compound of the invention, a pharmaceutically acceptable salt thereof or stereoisomer thereof, X is

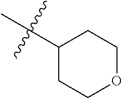 [SMILES: *C1CCOCC1]

and Y is phenyl,

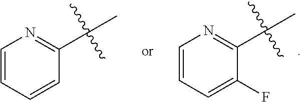 [SMILES: *c1ccccn1 ; *c1ncccc1F]

In one embodiment of the compound of the invention, a pharmaceutically acceptable salt thereof or stereoisomer thereof, Z is selected from hydrogen, —F, —Cl, —OH, methyl, ethyl, propyl, isopropyl, methoxy, ethoxy, propoxy or isopropoxy. In one embodiment of the compound of the invention, a pharmaceutically acceptable salt thereof or stereoisomer thereof, Z is hydrogen or methyl. In one embodiment of the compound of the invention, a pharmaceutically acceptable salt thereof or stereoisomer thereof, Z is hydrogen.

In one embodiment of the compound of the invention, a pharmaceutically acceptable salt thereof or stereoisomer thereof,

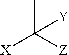

is selected from

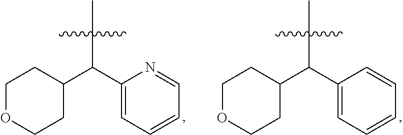

in which pyridine ring and benzene ring is independently optionally substituted with 1 substituent, and said substituent at each occurrence is selected from —F, —Cl or methyl. In one embodiment of the compound of the invention, a pharmaceutically acceptable salt thereof or stereoisomer thereof,

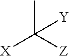

is selected from

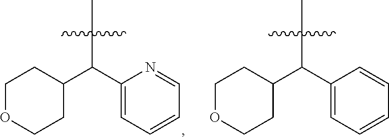

in which pyridine ring is optionally substituted with 1 substituent, and said substituent at each occurrence is selected from —F.

In a preferred embodiment, the present invention provides a compound of formula I, wherein Q is N.

In a more preferred embodiment, the present invention provides a compound of formula I, wherein Q is N, X is tetrahydropyranyl, such as, tetrahydropyran 4-yl, Y is phenyl, pyridyl, such as pyridin-2-yl, or pyridyl substituted with F, such as 3-fluoropyridin-2-yl, and Z is H.

In a further more preferred embodiment, the present invention provides a compound of formula I, wherein the compound is selected from the following:

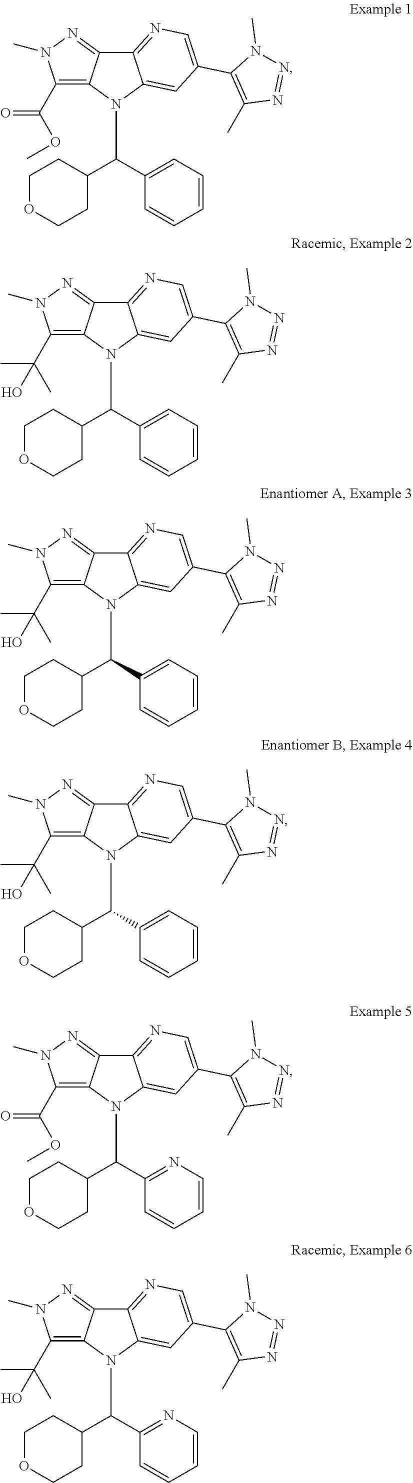 [SMILES: COC(=O)c1c2c(nn1C)c1ncc(-c3c(C)nnn3C)cc1n2C(c1ccccc1)C1CCOCC1 ; Cc1nnn(C)c1-c1cnc2c3nn(C)c(C(C)(C)O)c3n(C(c3ccccc3)C3CCOCC3)c2c1 ; Cc1nnn(C)c1-c1cnc2c3nn(C)c(C(C)(C)O)c3n([C@@H](c3ccccc3)C3CCOCC3)c2c1 ; Cc1nnn(C)c1-c1cnc2c3nn(C)c(C(C)(C)O)c3n([C@H](c3ccccc3)C3CCOCC3)c2c1 ; COC(=O)c1c2c(nn1C)c1ncc(-c3c(C)nnn3C)cc1n2C(c1ccccn1)C1CCOCC1 ; Cc1nnn(C)c1-c1cnc2c3nn(C)c(C(C)(C)O)c3n(C(c3ccccn3)C3CCOCC3)c2c1]

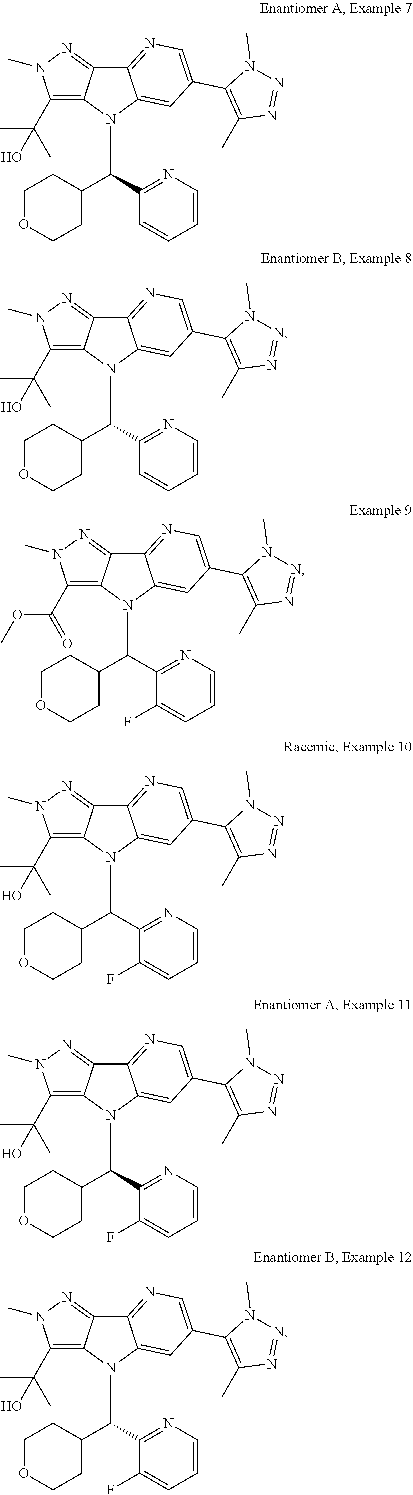

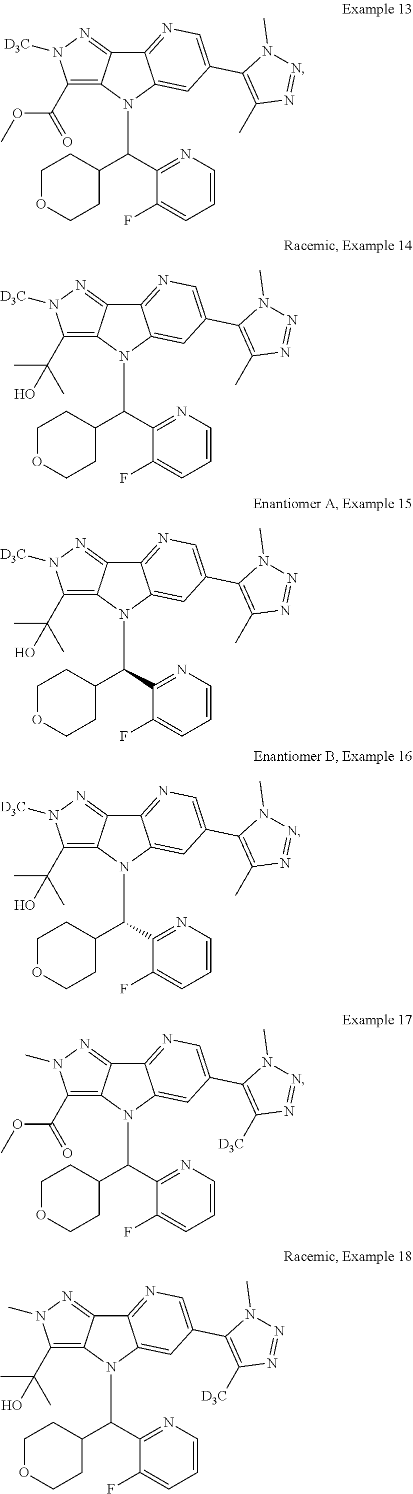 [SMILES: [2H]C([2H])([2H])n1nc2c3ncc(-c4c(C)nnn4C)cc3n(C(c3ncccc3F)C3CCOCC3)c2c1C(=O)OC ; [2H]C([2H])([2H])n1nc2c3ncc(-c4c(C)nnn4C)cc3n(C(c3ncccc3F)C3CCOCC3)c2c1C(C)(C)O ; [2H]C([2H])([2H])n1nc2c3ncc(-c4c(C)nnn4C)cc3n([C@@H](c3ncccc3F)C3CCOCC3)c2c1C(C)(C)O ; [2H]C([2H])([2H])n1nc2c3ncc(-c4c(C)nnn4C)cc3n([C@H](c3ncccc3F)C3CCOCC3)c2c1C(C)(C)O ; [2H]C([2H])([2H])c1nnn(C)c1-c1cnc2c3nn(C)c(C(=O)OC)c3n(C(c3ncccc3F)C3CCOCC3)c2c1 ; [2H]C([2H])([2H])c1nnn(C)c1-c1cnc2c3nn(C)c(C(C)(C)O)c3n(C(c3ncccc3F)C3CCOCC3)c2c1]

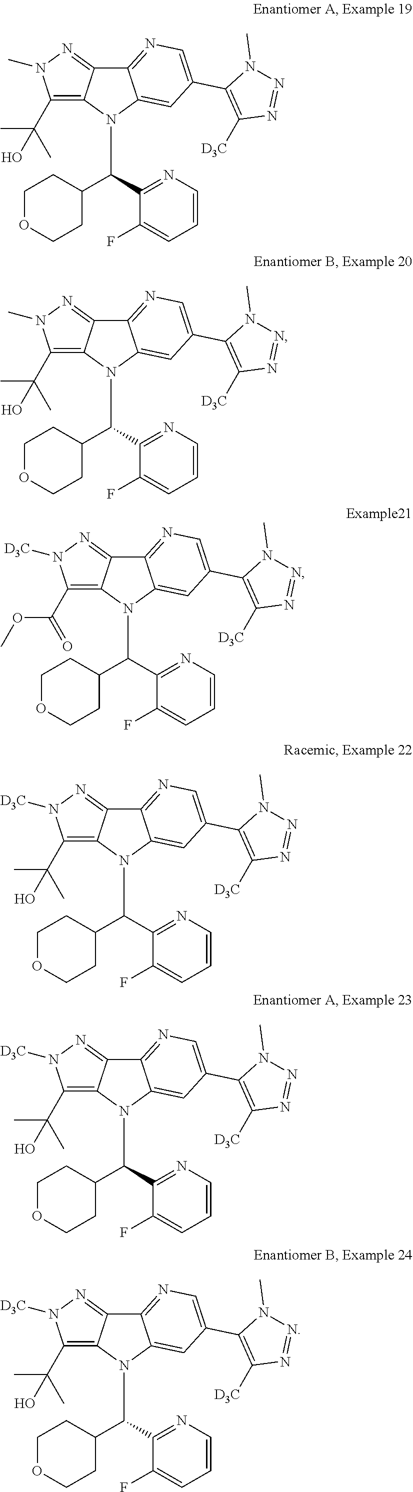

In an even further more preferred embodiment, the present invention provides a compound of formula I, wherein the compound is selected from the following:

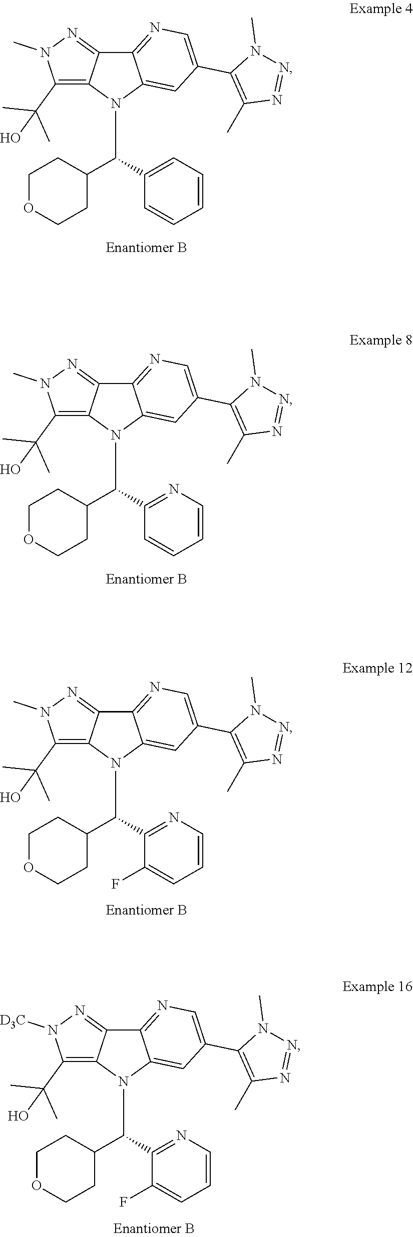 [SMILES: Cc1nnn(C)c1-c1cnc2c3nn(C)c(C(C)(C)O)c3n([C@H](c3ccccc3)C3CCOCC3)c2c1 ; Cc1nnn(C)c1-c1cnc2c3nn(C)c(C(C)(C)O)c3n([C@H](c3ccccn3)C3CCOCC3)c2c1 ; Cc1nnn(C)c1-c1cnc2c3nn(C)c(C(C)(C)O)c3n([C@H](c3ncccc3F)C3CCOCC3)c2c1 ; [2H]C([2H])([2H])n1nc2c3ncc(-c4c(C)nnn4C)cc3n([C@H](c3ncccc3F)C3CCOCC3)c2c1C(C)(C)O]

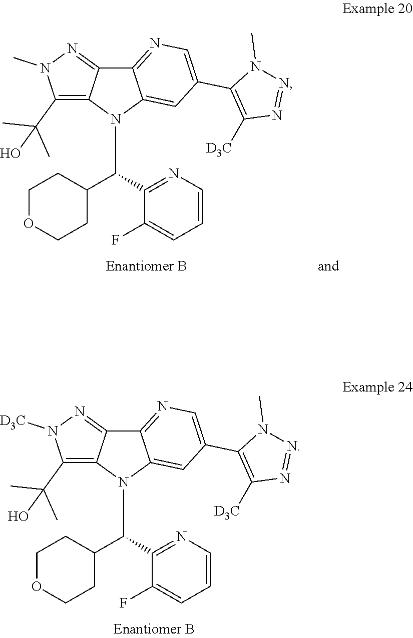 [SMILES: [2H]C([2H])([2H])c1nnn(C)c1-c1cnc2c3nn(C)c(C(C)(C)O)c3n([C@H](c3ncccc3F)C3CCOCC3)c2c1 ; [2H]C([2H])([2H])c1nnn(C)c1-c1cnc2c3nn(C([2H])([2H])[2H])c(C(C)(C)O)c3n([C@H](c3ncccc3F)C3CCOCC3)c2c1]

In the most preferred embodiment, the present invention provides a compound of formula I, wherein the compound is selected from the following:

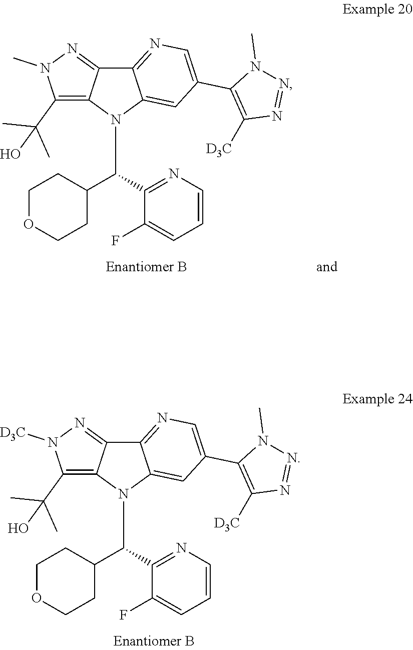 [SMILES: [2H]C([2H])([2H])c1nnn(C)c1-c1cnc2c3nn(C)c(C(C)(C)O)c3n([C@H](c3ncccc3F)C3CCOCC3)c2c1 ; [2H]C([2H])([2H])c1nnn(C)c1-c1cnc2c3nn(C([2H])([2H])[2H])c(C(C)(C)O)c3n([C@H](c3ncccc3F)C3CCOCC3)c2c1]

The compound of the present invention, a pharmaceutically acceptable salt thereof or stereoisomer thereof has an IC50 of less than 500 nM in the BRD4 (BD1) binding assay. The compound in the preferred embodiment of the present invention has an IC50 of less than 100 nM in the BRD4 (BD1) binding assay. The compound in the more preferred embodiment of the present invention has an IC50 of less than 50 nM in the BRD4 (BD1) binding assay. The compound in the further more preferred embodiment of the present invention has an IC50 of less than 10 nM in the BRD4 (BD1) binding assay. The compound in the even further more preferred embodiment of the present invention has an IC50 of less than 0.5 nM in the BRD4 (BD1) binding assay. The compound in the most preferred embodiment of the present invention has an IC50 of less than 0.2 nM, such as 0.17 nM in the BRD4 (BD1) binding assay.

In the second aspect of the present invention, there is provided a pharmaceutical composition which comprises a compound of the present invention, or a stereoisomer, or a pharmaceutically acceptable salt thereof and one or more pharmaceutically acceptable carriers, diluents or excipients.

In the third aspect of the present invention, there is provided the use of a compound of the present invention, a pharmaceutically acceptable salt thereof or stereoisomer thereof, in the manufacture of a medicament for the treatment of diseases or conditions for which a bromodomain inhibitor is indicated.

In the fourth aspect of the present invention, there is provided a method for inhibiting a bromodomain which comprises contacting the bromodomain with a compound of the present invention, a pharmaceutically acceptable salt thereof or stereoisomer thereof.

In the fifth aspect of the present invention, there is provided a method of treating cancer comprising administering a therapeutically effective amount of one or more compounds of the present invention or a pharmaceutically acceptable salt thereof or stereoisomer thereof.

In the sixth aspect of the present invention, there is provided a method for making a compound of the present invention or a stereoisomer, a tautomer, or a pharmaceutically acceptable salt thereof.

### Therapeutic Applications

The compound of the invention, a pharmaceutically acceptable salt thereof or stereoisomer thereof is bromodomain inhibitors and has potential utility in the treatment of diseases and conditions for which a bromodomain inhibitor is indicated.

In one embodiment, there is provided a method for the treatment of a disease or condition, for which a bromodomain inhibitor is indicated, in a subject in need thereof which comprises administering a therapeutically effective amount of compound of the present invention or a pharmaceutically acceptable salt thereof.

In one embodiment, there is provided a method for inhibiting a bromodomain which comprises contacting the bromodomain with a compound of the present invention or a pharmaceutically acceptable salt thereof.

While it is possible that for use in therapy, a compound of the present invention as well as pharmaceutically acceptable salts thereof may be administered as the compound itself, it is more commonly presented as a pharmaceutical composition.

### Definitions

Unless specifically stated otherwise herein, references made in the singular may also include the plural. For example, “a” and “an” may refer to either one, or one or more.

Unless otherwise indicated, any heteroatom with unsatisfied valences is assumed to have hydrogen atoms sufficient to satisfy the valences.

Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the invention. Many geometric isomers of C═C double bonds, C═N double bonds, ring systems, and the like can also be present in the compounds, and all such stable isomers are contemplated in the present invention. Cis- and trans- (or E- and Z-) geometric isomers of the compounds of the present invention are described and may be isolated as a mixture of isomers or as separated isomeric forms. The present compounds can be isolated in optically active or racemic forms. Optically active forms may be prepared by resolution of racemic forms or by synthesis from optically active starting materials. All processes used to prepare compounds of the present invention and intermediates made therein are considered to be part of the present invention. When enantiomeric or diastereomeric products are prepared, they may be separated by conventional methods, for example, by chromatography or fractional crystallization. Depending on the process conditions the end products of the present invention are obtained either in free (neutral) or salt form. Both the free form and the salts of these end products are within the scope of the invention. If so desired, one form of a compound may be converted into another form. A free base or acid may be converted into a salt; a salt may be converted into the free compound or another salt; a mixture of isomeric compounds of the present invention may be separated into the individual isomers. Compounds of the present invention, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the invention.

The present invention includes compounds described can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof.

The present invention includes all stereoisomers of the compound and pharmaceutically acceptable salts thereof. Further, mixtures of stereoisomers as well as isolated specific stereoisomers are also included. During the course of the synthetic procedures used to prepare such compounds or in using racemization or epimerization procedures known to those skilled in the art, the products of such procedures can be a mixture of stereoisomers.

The term “stereoisomer” as used in the present invention refers to an isomer in which atoms or groups of atoms in the molecule are connected to each other in the same order but differ in spatial arrangement, including conformational isomers and configuration isomers. The configuration isomers include geometric isomers and optical isomers, and optical isomers mainly include enantiomers and diastereomers.

When a substituent is noted as “optionally substituted”, the substituents are selected from, for example, substituents such as alkyl, cycloalkyl, aryl, heterocyclo, halo, hydroxy, alkoxy, oxo, alkanoyl, aryloxy, alkanoyloxy, amino, alkylamino, arylamino, arylalkylamino, disubstituted amines in which the 2 amino substituents are selected from alkyl, aryl or arylalkyl; alkanoylamino, aroylamino, aralkanoylamino, substituted alkanoylamino, substituted arylamino, substituted aralkanoylamino, thiol, alkylthio, arylthio, arylalkylthio, alkylthiono, arylthiono, arylalkylthiono, alkylsulfonyl, arylsulfonyl, arylalkylsulfonyl, sulfonamido, e.g. —SO₂NH₂, substituted sulfonamido, nitro, cyano, carboxy, carbamyl, e.g. —CONH₂, substituted carbamyl e.g. —CONHalkyl, —CONHaryl, —CONHarylalkyl or cases where there are two substituents on the nitrogen selected from alkyl, aryl or arylalkyl; alkoxycarbonyl, aryl, substituted aryl, guanidino, heterocyclyl, e.g., indolyl, imidazolyl, furyl, thienyl, thiazolyl, pyrrolidyl, pyridyl, pyrimidyl, pyrrolidinyl, piperidinyl, morpholinyl, piperazinyl, homopiperazinyl and the like, and substituted heterocyclyl, unless otherwise defined.

For purposes of clarity and in accordance with standard convention in the art, the symbol

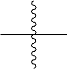

is used in formulas and tables to show the bond that is the point of attachment of the moiety or substituent to the core/nucleus of the structure.

Additonally, for purposes of clarity, where a substituent has a dash (-) that is not between two letters or symbols; this is used to indicate a point of attachment for a substituent. For example, —CONH₂ is attached through the carbon atom.

Additionally, for purposes of clarity, when there is no substituent shown at the end of a solid line, this indicates that there is a methyl (CH₃) group connected to the bond.

As used herein, the term “alkyl” or “alkylene” is intended to include both branched and straight-chain saturated aliphatic hydrocarbon groups having the specified number of carbon atoms. For example, “C₁-C₆ alkyl” denotes alkyl having 1 to 6 carbon atoms. Example alkyl groups include, but are not limited to, methyl (Me), ethyl (Et), propyl (e.g., n-propyl and isopropyl), butyl (e.g., n-butyl, isobutyl, t-butyl), and pentyl (e.g., n-pentyl, isopentyl, neopentyl).

The term “alkenyl” denotes a straight- or branch-chained hydrocarbon radical containing one or more double bonds and typically from 2 to 20 carbon atoms in length. For example, “C₂-C₈ alkenyl” contains from two to eight carbon atoms. Alkenyl groups include, but are not limited to, for example, ethenyl, propenyl, butenyl, 1-methyl-2-buten-1-yl, heptenyl, octenyl and the like.

The term “alkynyl” denotes a straight- or branch-chained hydrocarbon radical containing one or more triple bonds and typically from 2 to 20 carbon atoms in length. For example, “C₂-C₈ alkenyl” contains from two to eight carbon atoms. Representative alkynyl groups include, but are not limited to, for example, ethynyl, 1-propynyl, 1-butynyl, heptynyl, octynyl and the like.

The term “alkoxy” or “alkyloxy” refers to an —O-alkyl group. “C₁₋₆ alkoxy” (or alkyloxy), is intended to include C₁, C₂, C₃, C₄, C₅, and C₆ alkoxy groups. Example alkoxy groups include, but are not limited to, methoxy, ethoxy, propoxy (e.g., n-propoxy and isopropoxy), and t-butoxy.

The term “aryl”, as used herein, unless otherwise indicated, refers to an unsubstituted or substituted mono- or polycyclic aromatic ring system containing carbon ring atoms. The preferred aryls are mono cyclic or bicyclic 6-10 membered aromatic ring systems. Phenyl and naphthyl are preferred aryls. The most preferred aryl is phenyl.

The term “heterocyclic”, as used herein, unless otherwise indicated, refers to unsubstituted and substituted mono- or polycyclic non-aromatic ring system containing one or more heteroatoms. Preferred heteroatoms include N, O, and S, including N-oxides, sulfur oxides, and dioxides. Preferably the ring is three to eight membered and is either fully saturated or has one or more degrees of unsaturation. Multiple degrees of substitution, preferably one, two or three, are included within the present definition.

Examples of such heterocyclic groups include, but are not limited to azetidinyl, pyrrolidinyl, piperidinyl, piperazinyl, oxopiperazinyl, oxopiperidinyl, oxoazepinyl, azepinyl, tetrahydrofuranyl, dioxolanyl, tetrahydroimidazolyl, tetrahydrothiazolyl, tetrahydrooxazolyl, tetrahydropyranyl, morpholinyl, thiomorpholinyl, thiamorpholinyl sulfoxide, thiamorpholinyl sulfone and oxadiazolyl.

The term “heteroaryl”, as used herein, unless otherwise indicated, represents an aromatic ring system containing carbon (s) and at least one heteroatom. Heteroaryl may be monocyclic or polycyclic, substituted or unsubstituted. A monocyclic heteroaryl group may have 1 to 4 heteroatoms in the ring, while a polycyclic heteroaryl may contain 1 to 10 hetero atoms. A polycyclic heteroaryl ring may contain fused, spiro or bridged ring junction, for example, bycyclic heteroaryl is a polycyclic heteroaryl. Bicyclic heteroaryl rings may contain from 8 to 12 member atoms. Monocyclic heteroaryl rings may contain from 5 to 8 member atoms (cabons and heteroatoms). Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyl adeninyl, quinolinyl or isoquinolinyl.

The term “carbocyclic” refers to a substituted or unsubstituted monocyclic, bicyclic or polycyclic non-aromatic saturated ring, which optionally includes an alkylene linker through which the cycloalkyl may be attached. Examplary “cycloalkyl” groups includes but not limited to, cyclopropyl, cyclobutyl, cyclopentyl, cyclohexyl and so on.

“Halo” or “halogen” includes fluoro, chloro, bromo, and iodo. “Haloalkyl” is intended to include both branched and straight-chain saturated aliphatic hydrocarbon groups having the specified number of carbon atoms, substituted with 1 or more halogens. Examples of haloalkyl include, but are not limited to, fluoromethyl, difluoromethyl, trifluoromethyl, trichloromethyl, pentafluoroethyl, pentachloroethyl, 2,2,2-trifluoroethyl, heptafluoropropyl, and heptachloropropyl. Examples of haloalkyl also include “fluoroalkyl” that is intended to include both branched and straight-chain saturated aliphatic hydrocarbon groups having the specified number of carbon atoms, substituted with 1 or more fluorine atoms.

As referred to herein, the term “substituted” means that at least one hydrogen atom is replaced with a non-hydrogen group, provided that normal valencies are maintained and that the substitution results in a stable compound. Ring double bonds, as used herein, are double bonds that are formed between two adjacent ring atoms (e.g., C═C, C═N, or N═N).

When any variable occurs more than one time in any constituent or formula for a compound, its definition at each occurrence is independent of its definition at every other occurrence. Thus, for example, if a group is shown to be substituted with 0-3 R, then said group may optionally be substituted with up to three R groups, and at each occurrence R is selected independently from the definition of R. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom in which such substituent is bonded to the rest of the compound of a given formula, then such substituent may be bonded via any atom in such substituent. Combinations of substituents and/or variables are permissible only if such combinations result in stable compounds.

The present invention is intended to include all isotopes of atoms occurring in the present compounds. Isotopes include those atoms having the same atomic number but different mass numbers. By way of general example and without limitation, isotopes of hydrogen include deuterium and tritium. The isotopes of hydrogen can be denoted as ¹H (hydrogen), ²H (deuterium) and ³H (tritium). They are also commonly denoted as D for deuterium and T for tritium. In the application, CD₃ denotes a methyl group wherein all of the hydrogen atoms are deuterium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington: The Science and Practice of Pharmacy, ²²nd Edition, Allen, L. V. Jr., Ed.; Pharmaceutical Press, London, UK (2012), the disclosure of which is hereby incorporated by reference.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

### Methods of Preparation

The compounds in the present invention can be synthesized in a number of ways well to one skilled in the art of organic synthesis described below, together with synthetic methods known in the art of synthetic organic chemistry, or variations thereon as appreciated by those skilled in the art. Preferred methods are not limited as those described below. The references cited here are incorporated by reference in their entirety.

The methods of synthesis described hereinafter are intended as an illustration of the invention, without restricting its subject matter and the scope of the compounds claimed to these examples. Where the preparation of starting materials and reagents are not described, they are commercially obtainable or may be prepared analogously to known compounds or methods described herein. Substances described in the literature are prepared according to the published methods of synthesis. Compounds of formula I may be synthesized by reference to methods illustrated in the following schemes. As shown herein, the end compound is a product having the same structural formula depicted as formula I. It will be understood that any compound of formula I may be prepared by the selection of reagents with appropriate substitution. Solvents, temperature, pressures, and other reaction conditions may be readily selected by one of ordinary skill in the art. Protecting groups are manipulated according to standard methods of organic synthesis (T. W. Green and P. G. M. Wuts (1999) Protective Groups in Organic Synthesis, 3^{rd} edition, John Wiley &Sons). These groups are removed at certain stage of the compound synthesis using the methods that are apparent to those skilled in the art.

Compounds of Formula I may be prepared by reference to the methods illustrated in the following Schemes. As shown therein the end product is a compound having the same structural formula as Formula I. It will be understood that any compound of Formula I may be produced by the schemes by the suitable selection of reagents with appropriate substitution. Solvents, temperatures, pressures, and other reaction conditions may readily be selected by one of ordinary skill in the art. Starting materials are commercially available or readily prepared by one of ordinary skill in the art. Constituents of compounds are as defined herein or elsewhere in the specification.

As depicted in Scheme 1, Suzuki coupling of pyrazole 1 with the aromatic heterocycle 2, such as 2, 5-dibromo-3-nitropyridine using a suitable coupling catalyst, such as Pd (dppf)Cl₂ at the present of a base, like K₃PO₄ in THF/H₂O (5:1 volume ratio) can give the 3. A coupling of 3 with 4 (where M is a suitable coupling partner, such as boronic acid, boronic ester or stannane) by a Suzuki or Stille reaction can generate 5. Pyrazole ring of 5 is substituted by X₃, thereby giving the compound 6. NO₂ in 6 is reduced to NH₂, thereby giving the compound 7. Buchwald reaction of 7 using ligand, palladium catalyst and Cs₂CO₃ provides 8. Mitsunobu reaction of 8 with an alkylating agent 9 using triphenophosphine and diisopropyl azodicarboxylate (DIAD) provides 10.

As depicted in Scheme 2, the 10 can be generated from a reaction between 8 and an alkylating agent 11, where L is a leaving group such as a halide, mesylate or triflate, in the presence of a base, such as cesium carbonate.

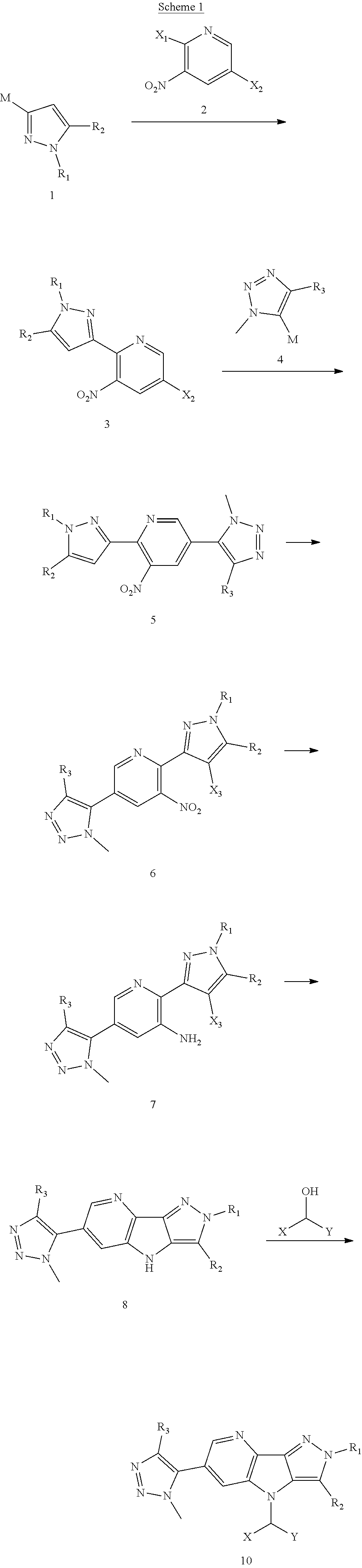

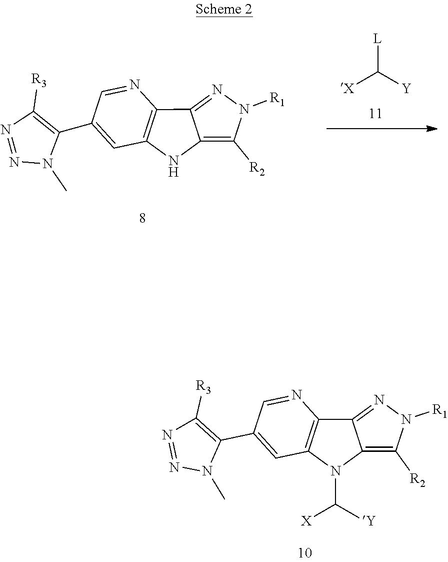

Note: The definitions of R₁ and R₂ in Schemes 1 and 2 are the same as those of R¹ and R² herein.

### EXAMPLES

The invention is further defined in the following Examples. It should be understood that the Examples are given by way of illustration only. From the above discussion and the Examples, one skilled in the art can ascertain the essential characteristics of the invention, and without departing from the spirit and scope thereof, can make various changes and modifications to adapt the invention to various uses and conditions. As a result, the invention is not limited by the illustrative examples set forth herein below, but rather is defined by the claims appended hereto.

The following table shows the part abbreviation of the present invention:

| | | | |
|---|---|---|---|
| aq | aqueous | EtOH | ethanol |
| CuI | Copper iodide | g | gram(s) |
| DCM | dichloromethane | h | hours |
| DIAD | diisopropyl | HPLC | high pressure liquid |
| | azodicarboxylate | | chromatography |
| DMF | dimethylformamide | LC-MS | Liquid chromatography- |
| DMSO | cimethyl sulfoxide | | mass spectroscopy |
| Equiv | equivalent(s) | Me | methane |
| Et₃N | triethylamine | ACN | acetonitrile |
| Et₂O | diethyl ether | min | minute(s) |
| EtOAc | ethyl acetate | mL | millimolar |
| Pd(dppf)Cl₂ | [1,1′- | NH₄Cl | ammonium chloride |
| | Bis(diphenylphosphino)ferr | CuBr | cuprous bromide |
| | ocene]dichloropalladium(II) | Na₂SO₄ | sodium sulfate |
| Prep-TLC | prep thin layer | I₂ | Iodine |
| | chromatography | KOAc | potassium Acetate |
| TEA | triethylamine | Xantphos | 4,5- |
| THF | tetrahydrofuran | | Bis(diphenylphosphino)- |
| PE | petroleum ether | | 9,9-diMethylxanthene |

### Intermediate Preparation

Unless otherwise stated, starting materials for the preparation of intermediates and Examples are commercially available.

### Intermediate-1

### Methyl 3-bromo-1-methyl-1H-pyrazole-5-carboxylate

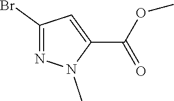

Step 1: Methyl 3-nitro-1H-pyrazole-5-carboxylate

To a solution of 3-nitro-1H-pyrazole-5-carboxylic acid (450 g, 2.86 mol) in MeOH (4.5 L) was added thionyl chloride (681.3 g, 5.73 mol) at 50-60° C. under N₂ atmosphere. The reaction mixture was stirred at 50° C. for 16 h. The reaction solution was cooled to 25° C. and concentrated to afford the title product (477 g, 97.4% yield) as a pale yellow solid. ¹H NMR (400 MHz, DMSO-d₆) δ7.51 (s, 1H), 3.90 (s, 3H).

Step 2: Methyl 1-methyl-3-nitro-1H-pyrazole-5-carboxylate

To a solution of methyl 3-nitro-1H-pyrazole-5-carboxylate (270 g, 1.58 mol) in DMF (1.89 L) was added K₂CO₃ (435 g, 3.15 mol). The mixture was cooled to 5° C., CH₃I (291 g, 2.05 mol) was added drop wise to the mixture and control the reaction temperature below 10° C. After adding drop wise, the reaction mixture was stirred at room temperature overnight. The reaction was diluted with water and extracted with DCM. The organic layer was washed with brine, dried over Na₂SO₄ and concentrated. The residue was purified by re-crystallization with MeOH to afford the title product (144 g, 51% yield) as a white solid. ¹H NMR (400 MHz, DMSO-d₆) δ7.55 (s, 1H), 4.19 (s, 3H), 3.89 (s, 3H).

Step 3: Methyl 3-amino-1-methyl-1H-pyrazole-5-carboxylate

To a solution of methyl 1-methyl-3-nitro-1H-pyrazole-5-carboxylate (144 g, 778 mmol) in MeOH (1.5 L) was added Pd/C (10% wt, 14.4 g). The reaction mixture was stirred at room temperature for 12 h under an atmosphere of hydrogen. The reaction mixture was filtered and the filtrate was concentrated to afford the title product (110 g, 92% yield) as a yellow solid. ¹H NMR (400 MHz, DMSO) δ5.97 (s, 1H), 4.81 (s, 2H), 3.84 (s, 3H), 3.77 (s, 3H).

Step 4: Methyl 3-bromo-1-methyl-1H-pyrazole-5-carboxylate

To a solution of methyl 3-amino-1-methyl-1H-pyrazole-5-carboxylate (110 g, 709 mmol) in ACN (1.65 L) was added CuBr (136 g, 638 mmol). The mixture was cooled to 0° C., tert-butyl nitrite (121 g 90% purity, 1055 mmol) was added drop wise. Then the reaction mixture was stirred at 0° C. for 2 h, and it was diluted with water and extract with DCM. The organic layer was washed with brine, dried over Na₂SO₄ and concentrated. The residue was purified by silica gel column to afford the title product (110 g, 71% yield) as a yellow oil. ¹H NMR (400 MHz, CDCl₃) δ6.81 (s, 1H), 4.16 (d, J=2.6 Hz, 3H), 3.89 (s, 3H).

### Intermediate-2

### Methyl 3-bromo-1-(methyl-d3)-1H-pyrazole-5-carboxylate

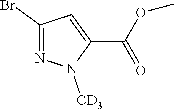

Step 1: Methyl 1-(methyl-d3)-3-nitro-1H-pyrazole-5-carboxylate

To a solution of methyl 3-nitro-1H-pyrazole-5-carboxylate (5 g, 29.22 mmol) in DMF (35 mL) was added K₂CO₃ (8.06 g, 58.44 mmol). The mixture was cooled to 5° C., CD₃I (5.5 g, 37.9 8 mmol) was added drop wise and the reaction's temperature was maintained below 10° C. After adding, the reaction mixture was warmed to room temperature, and stirred at room temperature overnight. Water (140 ml) was added to the solution, and extracted with DCM (10 mL)*3. The organic layer was washed with brine (5 mL)*5, dried over Na₂SO₄, and evaporated to give a residue. The residue was purified by recrystallization with MeOH to afford the title product (2 g, 37% yield) as a white solid. ¹H NMR (400 MHz, CDCl₃) δ7.382 (s, 1H), 3.943 (s, 3H).

Step 2: Methyl 3-amino-1-(methyl-d3)-1H-pyrazole-5-carboxylate

To a solution of Methyl 1-(methyl-d3)-3-nitro-1H-pyrazole-5-carboxylate (5 g, 26.60 mmol) in MeOH (100 mL) was added Pd/C (10% wt, 0.5 g). The reaction solution was stirred at room temperature under an atmosphere of hydrogen for 12 h. The reaction mixture was filtered and the filtrate was concentrated in vacuum to give the title product (3.8 g, 90.4%) as yellow solid. ¹H NMR (400 MHz, CDCl₃) δ6.121 (s, 1H), 3.832 (s, 3H), 3.139 (s, 2H).

Step 3: Methyl 3-bromo-1-(methyl-d3)-1H-pyrazole-5-carboxylate

To a solution of Methyl 3-amino-1-(methyl-d3)-1H-pyrazole-5-carboxylate (5 g, 31.64 mmol) in ACN (175 mL) was added CuBr₂ (II) (6.06 g, 27.17 mmol). The mixture was cooled to 0° C., tert-butyl nitrite (5.45 g, 90%), 47.56 mmol) was added drop wise at −5° C. and the reaction mixture was stirred at 0° C. for 2 h. The reaction mixture was quenched with water and extracted with DCM. The organic layer was washed with brine, dried over Na₂SO₄ and evaporated to give a residue. The residue was purified by silica gel column to afford the title product (4.25 g) as light-yellow oil and used to the next step directly. ¹H NMR (400 MHz, CDCl₃) δ6.080 (s, 1H), 3.885 (s, 3H).

### Example 1

### Methyl 6-(1,4-dimethyl-1H-1,2,3-triazol-5-yl)-2-methyl-4-(phenyl(tetrahydro-2H-pyran-4-yl)methyl)-2,4-dihydropyrazolo[3′,4′:4,5]pyrrolo[3,2- b]pyridine-3-carboxylate

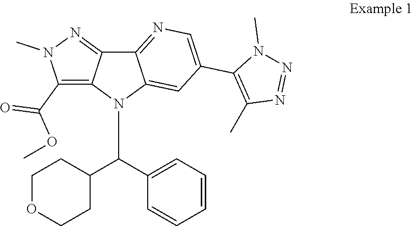 [SMILES: COC(=O)c1c2c(nn1C)c1ncc(-c3c(C)nnn3C)cc1n2C(c1ccccc1)C1CCOCC1]

### Step 1:

Methyl 1-methyl-3-(4,4,5,5-tetramethyl-1,3,2-dioxaborolan-2-yl)-1H-pyrazole-5-carboxylate

To a solution of methyl 3-bromo-1-methyl-1H-pyrazole-5-carboxylate (110 g, 502 mmol) and 4,4,4′,4′,5,5,5′,5′-octamethyl-2,2′-bi(1,3,2-dioxaborolane) (140 g, 552 mmol) in dioxane (1.1 L) was added KOAc (148 g, 1506 mmol). The reaction mixture was purged with N₂ for about 5 min and Pd(dppf)Cl₂ (18.2 g, 251 mmol) was added. The reaction mixture was heated at 100° C. under N₂ atmosphere for 16 h. The reaction mixture was cooled to room temperature and filtered. The filtrate was concentrated to afford the title product (120 g) as a brown solid. LC-MS: [M+H]⁺=267.1.

Step 2: Methyl 3-(5-bromo-3-nitropyridin-2-yl)-1-methyl-1H-pyrazole-5-carboxylate

To a solution of methyl 1-methyl-3-(4,4,5,5-tetramethyl-1,3,2-dioxaborolan-2-yl)-1H-pyrazole-5-carboxylate (120 g, 411 mmol) and 2,5-dibromo-3-nitropyridine (93 g, 330 mmol) in THF (550 mL) and water (110 mL) was added K₃PO₄ (175 g, 823 mmol). The reaction mixture was purged with N₂ for 5 min and Pd(dppf)Cl₂ (30 g, 42 mmol) was added. The reaction mixture was heated at 90° C. for 3 h under N₂ atmosphere. The reaction mixture was filtered and the filtrate was concentrated. The residue was purified by silica gel column to afford the title product (62 g, 22% yield over two steps).

¹H NMR (400 MHz, CDCl₃) δ8.84 (d, J=1.9 Hz, 1H), 8.08 (d, J=1.9 Hz, 1H), 7.34 (s, 1H), 4.22 (s, 3H), 3.91 (s, 3H).
Step 3: 1,4-Dimethyl-5-(tributylstannyl)-1H-1,2,3-triazole

To a solution of 1,4-dimethyl-1H-1,2,3-triazole (15 g, 150 mmol) in THF (300 mL) was added n-BuLi (73.88 mL, 180 mmol) at −70° C., The mixture was stirred at −70° C. for 1 h. Then tributylchlorostannane (55.37 g, 180 mmol) was added to the reaction mixture at −70° C. The reaction mixture was warmed to −30° C. and stirred at −30° C. for 1 h. The reaction mixture was quenched with 1 M CeF₂ solution and sat. NH₄Cl solution, extracted with EtOAc. The organic layer was washed with brine, dried over Na₂SO₄ and concentrated. The residue was purified by silica gel column chromatography (PE: EtOAc=1:0˜20:1) to afford the title product (53 g, 88% yield) as a colorless oil. ¹H NMR (400 MHz, CDCl₃) δ4.02 (s, 3H), 2.35 (s, 3H), 1.50 (dt, J=11.8, 7.5 Hz, 6H), 1.34 (dt, J=14.6, 7.3 Hz, 6H), 1.28-1.17 (m, 6H), 0.89 (t, J=7.3 Hz, 9H). LC-MS: [M+H]⁺=388.2.

Step 4: Methyl 3-(5-(1,4-dimethyl-1H-1,2,3-triazol-5-yl)-3-nitropyridin-2-yl)-1-methyl-1H-pyrazole-5-carboxylate

To a solution of methyl 3-(5-bromo-3-nitropyridin-2-yl)-1-methyl-1H-pyrazole-5-carboxylate (10 g, 0.029 mol) and 1,4-dimethyl-5-(tributylstannyl)-1H-1,2,3-triazole (13.6 g, 350 mmol) in DMF (100 mL) were added Pd(PPh₃)₄ (2.2 g, 1.88 mmol) and CuI (0.84 g, 4.35 mmol) at room temperature. The mixture was heated at 95° C. for 3 h under N₂ atmosphere. The reaction mixture was quenched with water and extracted with DCM, the organic layer was washed with brine, dried over Na₂SO₄ and concentrated. The residue was purified by silica gel column chromatography (PE:DCM=1:1˜DCM:MeOH=100:1) to afford the title product (8.12 g, 81.7% yield) as a yellow solid. ¹H NMR (400 MHz, CDCl₃) δ8.78 (s, 1H), 8.15 (d, J=2.0 Hz, 1H), 7.91 (s, 3H), 7.43 (s, 1H), 4.25 (s, 3H), 4.05 (s, 3H), 3.94 (s, 3H), 2.40 (s, 3H). LC-MS: [M+H]⁺=358.1.

Step 5: Methyl 3-(5-(1,4-dimethyl-1H-1,2,3-triazol-5-yl)-3-nitropyridin-2-yl)-4-iodo-1-methyl-1H-pyrazole-5-carboxylate

To a solution of methyl 3-(5-(1,4-dimethyl-1H-1,2,3-triazol-5-yl)-3-nitropyridin-2-yl)-1-methyl-1H-pyrazole-5-carboxylate (3.0 g, 8.4 mmol) in ACN (150 mL) were added Ce(NH₄)₂(NO₃)₆ (2.7 g, 5.04 mmol) and I₂ (1.065 g, 4.2 mmol) under N₂ atmosphere. The reaction mixture was heated at 80° C. for 2 h. Then Ce(NH₄)₂(NO₃)₆ (2.7 g, 5.04 mmol) and I₂ (1.065 g, 4.2 mmol) were added to the reaction mixture under N₂ atmosphere. The reaction mixture was concentrated. The residue was diluted with DCM and water, the separated aqueous phase was extracted with DCM. The organic layer was washed with brine, dried over Na₂SO₄ and concentrated to afford the title product (3.5 g, 86% yield) as a yellow solid. ¹H NMR (400 MHz, CDCl₃) δ8.93 (d, J=1.9 Hz, 1H), 8.28 (d, J=1.9 Hz, 1H), 4.29 (s, 3H), 4.09 (s, 3H), 4.01 (s, 3H), 2.44 (s, 3H). LC-MS: [M+H]⁺=484.0.

Step 6: Methyl 3-(3-amino-5-(1,4-dimethyl-1H-1,2,3-triazol-5-yl)pyridin-2-yl)-4-iodo-1-methyl-1H-pyrazole-5-carboxylate

To a solution of methyl 3-(5-(1,4-dimethyl-1H-1,2,3-triazol-5-yl)-3-nitropyridin-2-yl)-4-iodo-1-methyl-1H-pyrazole-5-carboxylate (3.5 g, 7.24 mmol) in EtOH (160 mL) and water (20 mL) was added iron powder (3.2 g, 58 mmol) and NH₄Cl (4.6 g, 87 mmol). The reaction mixture was heated at 80° C. for 3 h. The reaction mixture was filtered and the filtrate was concentrated. The residue was diluted with DCM and water, the separated aqueous phase was extracted with DCM. The organic layer was washed with brine, dried over Na₂SO₄ and concentrated. The residue was purified by silica gel column chromatography (DCM:MeOH=80:1˜60:1) to afford the title product (2.35 g, 71.6% yield) as a yellow solid. ¹H NMR (400 MHz, CDCl₃) δ8.09 (s, 1H), 6.99 (s, 1H), 5.17 (s, 2H), 4.28 (s, 3H), 4.01 (s, 6H), 2.37 (s, 3H). LC-MS: [M+H]⁺=454. 1.

Step 7: Methyl 6-(1,4-dimethyl-1H-1,2,3-triazol-5-yl)-2-methyl-2,4-dihydropyrazolo[3′,4′:4,5]pyrrolo[3,2-b]pyridine-3-carboxylate

A solution of methyl 3-(3-amino-5-(1,4-dimethyl-1H-1,2,3-triazol-5-yl)pyridin-2-yl)-4-iodo-1-methyl-1H-pyrazole-5-carboxylate (100 mg, 0.183 mmol), Pd₂(dba)₃ (30 mg, 0.033 mmol), Xantphos (30 mg, 0.052 mmol) and Cs₂CO₃ (148 mg, 0.46 mmol) in toluene (16 mL) was heated at 160° C. by microwave for 3 h under N₂ atmosphere (18 batches). The reaction mixture was concentrated. The residue was purified by silica gel column chromatography (DCM:MeOH=80:1˜40:1) to afford the title product (488 mg, 37.8% yield, 45% purity) as a yellow solid. LC-MS: [M+H]⁺=326.2.

Step 8: Methyl 6-(1,4-dimethyl-1H-1,2,3-triazol-5-yl)-2-methyl-4-(phenyl(tetrahydro-2H-pyran-4-yl)methyl)-2,4-dihydropyrazolo[3′,4′:4,5]pyrrolo [3,2-b]pyridine-3-carboxylate

To a solution of methyl 6-(1,4-dimethyl-1H-1,2,3-triazol-5-yl)-2-methyl-2,4-dihydropyrazolo[3′,4′:4,5]pyrrolo[3,2-b]pyridine-3-carboxylate (500 mg, 1.52 mmol), phenyl(tetrahydro-2H-pyran-4-yl)methanol (140 mg, 0.77 mmol) and PPh₃ (980 mg, 3.8 mmol) in toluene (40 mL) was added DIAD (700 mg, 3.4 mmol) at 20° C. The reaction mixture was heated at 80° C. for 12 h. The reaction was concentrated. The residue was purified by prep-TLC (DCM:MeOH=20:1) to afford the title product (120 mg, 18% yield) as a yellow solid. ¹H NMR (400 MHz, CDCl₃) δ8.35 (s, 1H), 7.46˜7.42 (m, 3H), 7.36˜7.32 (m, 2H), 7.29 (d, J=7.6 Hz, 1H), 6.56 (d, J=10.4 Hz, 1H), 4.49 (s, 3H), 4.09 (s, 3H), 4.05˜4.01 (m, 1H), 3.88˜3.85 (m, 1H), 3.78 (s, 3H), 3.56˜3.50 (m, 1H), 3.35˜3.29 (m, 1H), 2.98˜2.90 (m, 1H), 2.20 (s, 3H), 2.04˜2.01 (m, 1H), 1.58˜1.42(m, 3H), 1.00˜0.97 (m, 1H). LC-MS: [M+H]⁺=500.3.

### Example 2, 3 & 4

### 2-(6-(1,4-Dimethyl-1H-1,2,3-triazol-5-yl)-2-methyl-4-(phenyl(tetrahydro-2H-pyran-4-yl)methyl)-2,4-dihydropyrazolo[3′,4′:4,5]pyrrolo[3,2-b]pyridin-3-yl)propan-2-ol

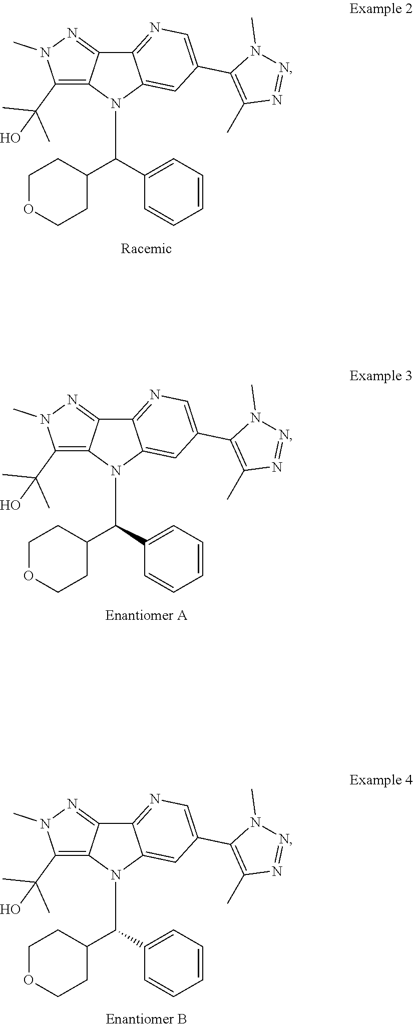 [SMILES: Cc1nnn(C)c1-c1cnc2c3nn(C)c(C(C)(C)O)c3n(C(c3ccccc3)C3CCOCC3)c2c1 ; Cc1nnn(C)c1-c1cnc2c3nn(C)c(C(C)(C)O)c3n([C@@H](c3ccccc3)C3CCOCC3)c2c1 ; Cc1nnn(C)c1-c1cnc2c3nn(C)c(C(C)(C)O)c3n([C@H](c3ccccc3)C3CCOCC3)c2c1]

Step 1: 2-(6-(1,4-Dimethyl-1H-1,2,3-triazol-5-yl)-2-methyl-4-(phenyl(tetrahydro-2H-pyran-4-yl)methyl)-2,4-dihydropyrazolo[3′,4′:4,5]pyrrolo[3,2-b]pyridin-3-yl)propan-2-ol

To a solution of methyl 6-(1,4-dimethyl-1H-1,2,3-triazol-5-yl)-2-methyl-4-(phenyl(tetrahydro-2H-pyran-4-yl)methyl)-2,4-dihydropyrazolo[3′,4′:4,5]pyrrolo[3,2-b]pyridine-3-carboxylate (120 mg, 0.24 mmol) in THF (1.2 mL) was added MeMgCl (12 mL, 12 mmol) at 0° C. The reaction mixture was stirred at 0° C. for 5 min. Then the reaction mixture was heated to 66° C. and stirred at 66° C. for 40 min. The reaction mixture was cooled to −10° C. The reaction mixture was quenched with sat NH₄Cl solution and extracted with DCM. The organic layer was washed with brine, dried over Na₂SO₄ and concentrated. The residue was purified by prep-TLC (DCM:MeOH=20:1) and prep-HPLC (Welch Ultimate XB-C18, 21.2*250 mm, 5 um, 30%-80% CH₃CN/water, 0.1% CF₃COOH) to afford the product (26.5 mg, 22% yield) as a yellow solid. ¹H NMR (400 MHz, CDCl₃) δ8.28 (s, 1H), 7.43˜7.40 (m, 2H), 7.32˜7.29 (m, 3H), 7.24˜7.19 (m, 1H), 6.64 (d, J=10.4 Hz, 1H), 4.27 (s, 3H), 4.02˜3.98 (m, 1H), 3.86˜3.83 (m, 1H), 3.74 (s, 3H), 3.54˜3.48(m, 1H), 3.31˜3.25 (m,1H), 2.88˜2.84 (m, 1H), 2.19 (s, 3H), 2.05˜2.01 (m, 1H), 1.94˜1.92 (m, 6H), 1.59˜1.50 (m, 2H), 0.91˜0.88 (m, 1H). LC-MS: [M+H]⁺=500.3. Racemic example 1 (25.1 mg) was separated by Chiral Prep SFC (column: Lux 5 um Cellulose-42 cm×25 cm, 5 um; Mobile phase: MeOH:EtOH=50:50; Flow rate: 25 mL/min) to give Enantiomer A example 2 (11.1 mg, yield 44.2%) and Enantiomer B example B (12.1 mg, yield 48.2%). Enantiomer A example 3: ¹H NMR (400 MHz, CDCl₃) δ8.35 (s, 1H), 7.43-7.29 (m, 5H), 6.68 (d, J=14 Hz, 1H), 4.28 (s, 3H), 4.03-3.99 (m, 1H), 3.87-3.83 (m, 1H), 3.75 (s, 3H), 3.56-3.48 (m, 1H), 3.33-3.25 (m, 1H), 2.93-2.81 (m, 1H), 2.19 (s, 3H), 2.07-2.03 (m, 1H), 1.95-1.93 (m, 6H), 1.61-1.51 (m, 2H), 0.89-0.85 (m, 1H). LC-MS: [M+H]⁺=500.3. Chiral SFC=2.499 min (Column: Lux Cellulose-4, 100*4.6 mm, 3 um H19-381245; Mobile Phase A: Ethanol; Mobile Phase B: Methanol; Flow: 1 mL/min; Detection: UV at 254 nm). Enantiomer B example 4: ¹H NMR (400 MHz, CDCl₃) δ8.35 (s, 1H), 7.43-7.29 (m, 5H), 6.68 (d, J=14 Hz, 1H), 4.28 (s, 3H), 4.03-3.99 (m, 1H), 3.87-3.83 (m, 1H), 3.75 (s, 3H), 3.56-3.48 (m, 1H), 3.33-3.25 (m, 1H), 2.93-2.81 (m, 1H), 2.19 (s, 3H), 2.07-2.03 (m, 1H), 1.95-1.93 (m, 6H), 1.61-1.51 (m, 2H), 0.89-0.85 (m, 1H). LC-MS: [M+H]⁺=500.3. Chiral SFC=2.926 min (Column: Lux Cellulose-4, 100*4.6 mm, 3 um H19-381245; Mobile Phase A: Ethanol; Mobile Phase B: Methanol; Flow: 1 mL/min; Detection: UV at 254 nm).

### Example 5

### Methyl 6-(1,4-dimethyl-1H-1,2,3-triazol-5-yl)-2-methyl-4-(pyridin-2-yl)tetrahydro-2H-pyran-4-yl)methyl)-2,4-dihydropyrazolo [3′,4′:4,5]pyrrolo [3,2- b]pyridine-3-carboxylate

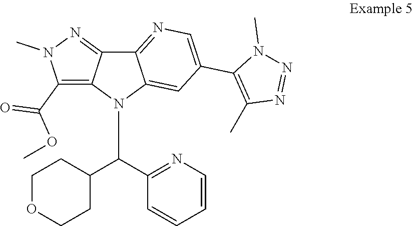 [SMILES: COC(=O)c1c2c(nn1C)c1ncc(-c3c(C)nnn3C)cc1n2C(c1ccccn1)C1CCOCC1]

### Step 1: Pyridin-2-yl(tetrahydro-2H-pyran-4-yl)methyl Methanesulfonate

To a solution of pyridin-2-yl(tetrahydro-2H-pyran-4-yl)methanol (5 g, 25 mmol) and Et₃N (5.4 mL, 35 mmol) in DCM (100 mL) was added MsCl (2.9 g, 26 mmol) at 0° C. under N₂ atmosphere. The reaction mixture was stirred at 0° C. for 2 h. The reaction mixture was quenched with sat NH₄Cl solution and extracted with DCM. The organic layer was washed with brine, dried over Na₂SO₄ and concentrated to afford the title product (6.4 g) as a brown solid.

### Step 2: Methyl 6-(1,4-dimethyl-1H-1,2,3-triazol-5-yl)-2-methyl-4-(pyridin-2-yl(tetrahydro-2H-pyran-4-yl)methyl)-2,4-dihydropyrazolo[3′,4′:4,5]pyrrolo[3,2-b]pyridine-3-carboxylate

To a solution of methyl 6-(1,4-dimethyl-1H-1,2,3-triazol-5-yl)-2-methyl-2,4-dihydropyrazolo[3′,4′:4,5]pyrrolo[3,2-b]pyridine-3-carboxylate (690 mg, 2.1 mmol) and pyridin-2-yl(tetrahydro-2H-pyran-4-yl)methyl methanesulfonate (630 mg, 2.3 mmol) in ACN (60 mL) was added Cs₂CO₃ (990 mg, 2.5 mmol) at 20° C. The reaction mixture was heated at 70° C. for 12 h. The reaction mixture was concentrated. The residue was purified by prep-TLC (DCM:MeOH=20:1) to afford the title product (200 mg, 18.9% yield) as a yellow solid. ¹H NMR (400 MHz, CDCl₃) δ8.60 (d, J=4.4 Hz, 1H), 8.48 (s, 1H), 8.39 (s, 1H), 7.66 (t, J=7.2 Hz, 1H), 7.45 (d, J=7.6 Hz, 1H), 7.23 (t, J=6.0 Hz, 1H), 6.87 (d, J=11.2 Hz, 1H), 4.46 (s, 3H), 4.12 (s, 3H), 4.00˜3.93 (m, 4H), 3.84˜3.81 (m, 1H), 3.50˜3.44 (m, 1H), 3.34˜3.25 (m, 2H), 2.37 (s, 3H), 1.44˜1.33 (m, 2H), 0.95˜0.80 (m, 2H). LC-MS: [M+H]⁺=501.3.

### Example 6, 7 & 8

### 2-(6-(1,4-Dimethyl-1H-1,2,3-triazol-5-yl)-2-methyl-4-(pyridin-2-yl)tetrahydro-2H-pyran-4-yl)methyl)-2,4-dihydropyrazolo[3′,4′:4,5]pyrrolo[3,2-b]pyridin-3-yl)propan-2-ol

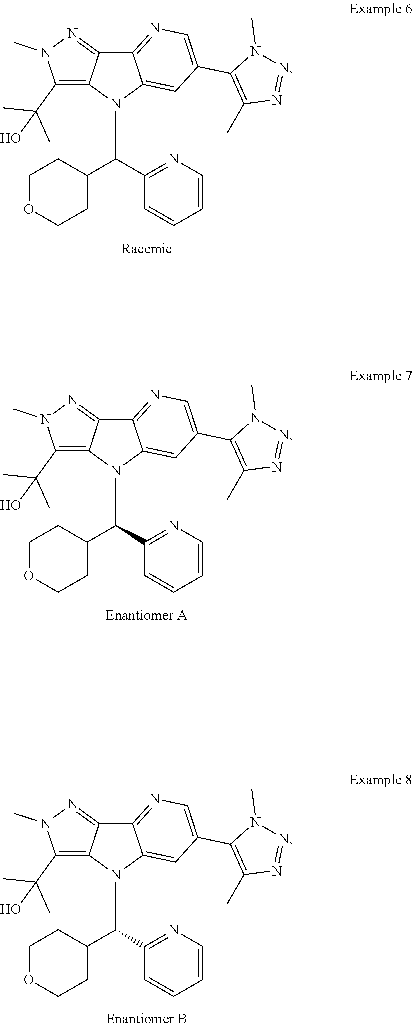 [SMILES: Cc1nnn(C)c1-c1cnc2c3nn(C)c(C(C)(C)O)c3n(C(c3ccccn3)C3CCOCC3)c2c1 ; Cc1nnn(C)c1-c1cnc2c3nn(C)c(C(C)(C)O)c3n([C@@H](c3ccccn3)C3CCOCC3)c2c1 ; Cc1nnn(C)c1-c1cnc2c3nn(C)c(C(C)(C)O)c3n([C@H](c3ccccn3)C3CCOCC3)c2c1]

Step 1: 2-(6-(1,4-Dimethyl-1H-1,2,3-triazol-5-yl)-2-methyl-4-(pyridin-2-yl)tetrahydro-2H-pyran-4-yl)methyl)-2,4-dihydropyrazolo[3′,4′:4,5]pyrrolo[3,2-b]pyridin-3-yl)propan-2-ol

To a solution of methyl 6-(1,4-dimethyl-1H-1,2,3-triazol-5-yl)-2-methyl-4-(pyridin-2-yl(tetrahydro-2H-pyran-4-yl)methyl)-2,4-dihydropyrazolo[3′,4′:4,5]pyrrolo[3,2-b]pyridine-3-carboxylate (200 mg, 0.41 mmol) in THF (4 mL) was added CH₃MgBr (20 mL, 20 mmol) at 0° C. The reaction mixture was stirred at 0° C. for 5 min. Then the reaction mixture was warmed to 66° C. and stirred at 66° C. for 40 min. The reaction mixture was cooled to −10° C. Then the mixture was quenched with sat NH₄Cl solution and extracted with DCM. The organic layer was washed with brine, dried over Na₂SO₄ and concentrated. The residue was purified by prep-TLC (DCM:MeOH=20:1) and prep-HPLC (Welch Ultimate XB-C18, 21.2*250 mm, 5 um, 25%-70% CH₃CN/water, 0.1% CF₃COOH) to afford the title product (65 mg, 32.5% yield) as a yellow solid. ¹H NMR (400 MHz, CDCl₃) δ8.50 (d, J=4.0 Hz, 1H), 8.25 (s, 1H), 8.05 (s, 1H), 7.60 (s, 1H), 7.43 (s, 1H), 7.15˜7.13 (m,1H), 6.57 (d, J=10.8 Hz, 1H), 4.17 (s, 3H), 3.90˜3.86 (m, 4H), 3.77˜3.73 (m, 1H), 3.41˜3.55 (m,1H), 3.20˜3.04 (m, 2H), 2.28 (s, 3H), 1.86˜1.83 (m, 6H), 1.31˜1.24 (m, 3H), 0.82˜0.76 (m, 1H). LC-MS: [M+H]⁺=501.3. Racemic example 4 (70.1 mg) was separated by Chiral Prep SFC (column: Lux 5 um Cellulose-4 2 cm×25 cm, 5 um; Mobile phase: MeOH:EtOH=50:50; Flow rate: 25 mL/min) to give Enantiomer A example 5 (24.4 mg, 34.8% yield) and Enantiomer B example 6 (29.4 mg, 41.9% yield). Enantiomer A example 7: ¹H NMR (400 MHz CDCl₃) δ8.59 (d, J=6.0 Hz, 1H), 8.34 (s, 1H), 8.17 (s, 1H), 7.60 (s, 1H), 7.43 (s, 1H), 7.15˜7.13 (m, 1H), 6.57 (d, J=10.8 Hz, 1H), 4.17 (s, 3H), 3.90˜3.86 (m, 4H), 3.77˜3.73 (m, 1H), 3.41˜3.55 (m, 1H), 3.20˜3.04 (m, 2H), 2.28 (s, 3H), 1.86˜1.83 (m, 6H), 1.31˜1.24 (m, 3H) 0.82˜0.76 (m, 1H). LC-MS: [M+H]⁺=501.3. Chiral SFC=2.788 min (Column: Lux Cellulose-4, 100*4.6 mm, 3 um H19-381245; Mobile Phase A: Ethanol; Mobile Phase B: Methanol; Flow: 1 mL/min; Detection: UV at 254 nm). Enantiomer B example 8: ¹H NMR (400 MHz, CDCl₃) δ8.50 (d, J=4.0 Hz, 1H), 8.25 (s, 1H), 8.05 (s, 1H), 7.60 (s, 1H), 7.43 (s, 1H), 7.15˜7.13 (m, 1H), 6.57 (d, J=10.8 Hz, 1H), 4.17 (s, 3H), 3.90˜3.86 (m, 4H), 3.77˜3.73 (m, 1H), 3.41˜3.55 (m, 1H), 3.20˜3.04 (m, 2H), 2.28 (s, 3H), 1.86˜1.83 (m, 6H), 1.31˜1.24 (m, 3H), 0.82˜0.76 (m, 1H). LC-MS: [M+H]⁺=501.3. Chiral SFC=3.568 min (Column: Lux Cellulose-4, 100*4.6 mm, 3 um H19-381245; Mobile Phase A: Ethanol; Mobile Phase B: Methanol; Flow: 1 mL/min; Detection: UV at 254 nm).

### Example 9

Methyl 6-(1,4-dimethyl-1H-1,2,3-triazol-5-yl)-4-((3-fluoropyridin-2-yl)(tetrahydro-2H-pyran-4-yl)methyl)-2-methyl-2,4-dihydropyrazolo[3′,4′:4,5]pyrrolo[3,2-b]pyridine-3-carboxylate

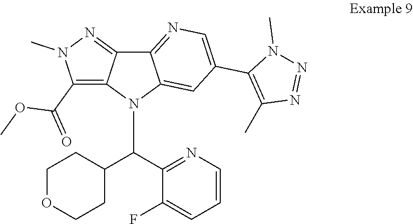 [SMILES: COC(=O)c1c2c(nn1C)c1ncc(-c3c(C)nnn3C)cc1n2C(c1ncccc1F)C1CCOCC1]

### Step 1: (3-Fluoropyridin-2-yl)(tetrahydro-2H-pyran-4-yl)methyl Methanesulfo Nate

To a solution of (3-fluoropyridin-2-yl)(tetrahydro-2H-pyran-4-yl)methanol (5 g, 24 mmol) and Et₃N (5.4 mL, 35 mmol) in DCM (100 mL) was added MsCl (2.9 g, 26 mmol) at 0° C. under N₂ atmosphere. The reaction mixture was stirred at 0° C. for 2 h. The reaction mixture was quenched with sat NH₄Cl and extracted with DCM. The organic layer was washed with brine, dried over Na₂SO₄ and concentrated to afford the title product (6.1 g) as a brown oil. ¹H NMR (400 MHz, CDCl₃) δ8.52 (d, J=3.5 Hz, 1H), 7.48 (t, J=8.9 Hz, 1H), 7.36 (dt, J=8.4, 4.3 Hz, 1H), 5.68 (d, J=9.0 Hz, 1H), 4.06 (d, J=8.5 Hz, 1H), 3.97-3.87 (m, 1H), 3.37 (dt, J=32.0, 11.6 Hz, 2H), 2.87 (s, 3H), 2.55-2.36 (m, 1H), 2.00 (d, J=13.3 Hz, 1H), 1.63-1.54 (m, 1H), 1.43 (tt, J=12.0, 6.0 Hz, 1H), 1.11 (d, J=13.0 Hz, 1H). LC-MS: [M+H]⁺=290.1.

Step 2: Methyl 6-(1,4-dimethyl-1H-1,2,3-triazol-5-yl)-4-((3-fluoropyridin-2-yl)(tetrahydro-2H-pyran-4-yl)methyl)-2-methyl-2,4-dihydropyrazolo[3′,4′:4,5]pyrrolo[3,2-b]pyridine-3-carboxylate

To a solution of methyl 6-(1,4-dimethyl-1H-1,2,3-triazol-5-yl)-2-methyl-2,4-dihydropyrazolo[3′,4′:4,5]pyrrolo[3,2-b]pyridine-3-carboxylate (540 mg, 1.6 mmol, 40% purity) and (3-fluoropyridin-2-yl)(tetrahydro-2H-pyran-4-yl)methyl methanesulfonate (58 mg, 0.201 mmol) in ACN (6 mL) was added Cs₂CO₃ (648 mg, 1.98 mmol). The reaction mixture was stirred at 70° C. for 12 h. The reaction mixture was concentrated. The residue was purified by prep-TLC (DCM:MeOH=20:1) to afford the title product (180 mg, 21% yield) as a yellow solid. ¹H NMR (400 MHz, CDCl₃) δ8.45 (d, J=4.4 Hz, 1H), 8.37 (s, 1H), 8.21 (s, 1H), 7.37-7.33 (m, 1H), 7.30-7.28 (m, 1H), 6.87 (d, J=10.8 Hz, 1H), 4.47 (s, 3H), 4.10 (s, 3H), 3.96 (s, 4H), 3.53˜3.40 (m, 1H), 3.37˜3.30 (m, 2H), 2.32 (s, 3H), 1.85˜1.76 (m, 2H), 0.88˜084 (m, 2H). LC-MS: [M+H]⁺=519.2.

### Example 10, 11 & 12

### 2-(6-(1,4-Dimethyl-1H-1,2,3-triazol-5-yl)-4-((3-fluoropyridin-2-yl)(tetrahydro-2H-pyran-4-yl)methyl)-2-methyl-2,4-dihydropyrazolo[3′,4′:4,5]pyrrolo[3,2-b]pyridin-3-yl)propan-2-ol

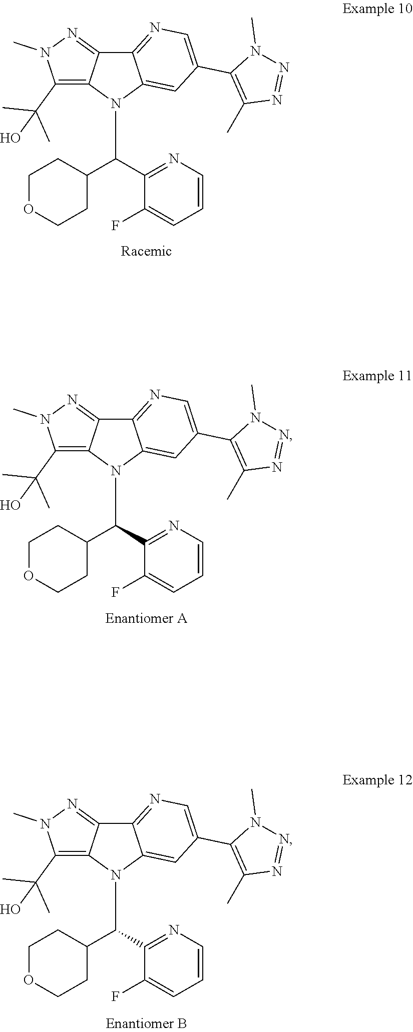 [SMILES: Cc1nnn(C)c1-c1cnc2c3nn(C)c(C(C)(C)O)c3n(C(c3ncccc3F)C3CCOCC3)c2c1 ; Cc1nnn(C)c1-c1cnc2c3nn(C)c(C(C)(C)O)c3n([C@@H](c3ncccc3F)C3CCOCC3)c2c1 ; Cc1nnn(C)c1-c1cnc2c3nn(C)c(C(C)(C)O)c3n([C@H](c3ncccc3F)C3CCOCC3)c2c1]

Step 1: 2-(6-(1,4-dimethyl-1H-1,2,3-triazol-5-yl)-4-((3-fluoropyridin-2-yl)(tetrahydro-2H-pyran-4-yl)methyl)-2-methyl-2,4-dihydropyrazolo[3′,4′:4,5]pyrrolo[3,2-b]pyridin-3-yl)propan-2-ol

To a solution of methyl 6-(1,4-dimethyl-1H-1,2,3-triazol-5-yl)-4-((3-fluoropyridin-2-yl)(tetrahydro-2H-pyran-4-yl)methyl)-2-methyl-2,4-dihydropyrazolo[3′,4′:4,5]pyrrolo[3,2-b]pyridine-3-carboxylate (180 mg, 0.3 mmol) in THF (0.5 mL) was added MeMgCl (36 mL, 36 mmol) at 0° C. The reaction mixture was stirred at 0° C. for 5 min. Then the reaction mixture was heated to 66° C. and stirred at 66° C. for 40 min. The reaction mixture was cooled to −10° C. Then the mixture was quenched with sat NH₄Cl solution and extracted with DCM. The organic layer was washed with brine, dried over Na₂SO₄ and concentrated. The residue was purified by prep-TLC (DCM:MeOH=20:1) and prep-HPLC (Welch Ultimate XB-C18, 21.2*250 mm, 5 um, 30%-80% CH₃CN/water, 0.1% CF₃COOH) to afford the title product (38 mg, 21% yield) as a yellow solid. ¹H NMR (400 MHz, CDCl₃) δ8.44 (d, J=4.4 Hz, 2H), 7.40 (t, J=8.8 Hz, 1H), 7.32˜7.29 (m, 1H), 7.09 (d, J=10.4 Hz, 1H), 4.27 (s, 3H), 4.05˜3.85 (m, 5H), 3.50˜3.44 (m, 1H), 3.31˜3.26 (m, 2H), 2.33 (s, 3H), 1.91 (s, 6H), 1.85˜1.76 (m, 1H), 1.71˜1.41 (m, 2H) 0.92˜088 (m, 1H). LC-MS: [M+H]⁺=519.3. Racemic example 10 (30 mg) was separated by Chiral Prep SFC (column: CHIRAL ART Cellulose-SB3 cm×25 cm, 5 um; Mobile phase: Hex:EtOH=70:30; Flow rate: 35 mL/min) to give Enantiomer A example 11 (11.4 mg, 38.0% yield) and Enantiomer B example 12 (12.8 mg, 42.7% yield). Enantiomer A example 11: ¹H NMR (400 MHz, CDCl₃) δ8.43 (d, J=6 Hz, 1H), 8.29 (d, J=2 Hz, 1H), 7.99 (d, J=2 Hz, 1H), 7.39-7.33 (m, 1H), 7.29-7.28 (m, 1H), 6.98 (d, J=16 Hz, 1H), 4.24 (s, 3H), 3.99-3.83 (m, 5H), 3.52-3.43 (m, 1H), 3.35-3.23 (m, 2H), 2.31 (s, 3H), 1.91 (d, J=6.4 Hz, 6H), 1.87˜1.79 (m, 2H), 1.73˜1.42 (m, 1H), 0.95˜0.88 (m, 1H). LC-MS: [M+H]⁺=519.3. Chiral SFC=4.426 min (Column: Lux Cellulose-4, 100*4.6 mm, 3 um H19-381245; Mobile Phase A: n-Hexane; Mobile Phase B: ethanol; Flow: 1 mL/min; Detection: UV at 254 nm). Enantiomer B example 12: ¹H NMR (400 MHz, CDCl₃) δ 8.43 (d, J=6 Hz, 1H), 8.29 (d, J=2 Hz, 1H), 7.99 (d, J=2 Hz, 1H), 7.39-7.33 (m, 1H), 7.29-7.28 (m, 1H), 6.98 (d, J=16 Hz, 1H), 4.24 (s, 3H), 3.99-3.83 (m, 5H), 3.52-3.43 (m, 1H), 3.35-3.23 (m, 2H), 2.31 (s, 3H), 1.91 (d, J=6.4 Hz, 6H), 1.87˜1.79 (m, 2H), 1.73˜1.42 (m, 1H), 0.95˜0.88 (m, 1H). LC-MS: [M+H]⁺=519.3. Chiral SFC=3.908 min (Column: Lux Cellulose-4, 100*4.6 mm, 3 um H19-381245; Mobile Phase A: n-Hexane; Mobile Phase B: ethanol; Flow: 1 mL/min; Detection: UV at 254 nm).

### Example 13

Methyl 6-(1,4-dimethyl-1H-1,2,3-triazol-5-yl)-4-((3-fluoropyridin-2-yl)(tetrahydro-2H-pyran-4-yl)methyl)-2-(methyl-d3)-2,4-dihydropyrazolo[3′,4′:4,5]pyrrolo[3,2-b]pyridine-3-carboxylate

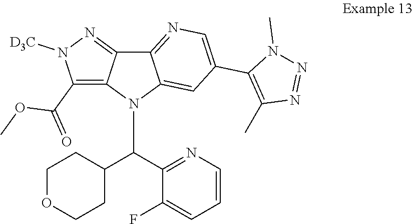 [SMILES: [2H]C([2H])([2H])n1nc2c3ncc(-c4c(C)nnn4C)cc3n(C(c3ncccc3F)C3CCOCC3)c2c1C(=O)OC]

Step 1: Methyl 1-(methyl-d3)-3-(4,4,5,5-tetramethyl-1,3,2-dioxaborolan-2-yl)-1H-pyrazole-5-carboxylate

To a solution of methyl 3-bromo-1-(methyl-d3)-1H-pyrazole-5-carboxylate (5 g, 22.52 mmol) and 4,4,4′,4′,5,5,5′,5′-octamethyl-2,2′-bi(1,3,2-dioxaborolane) (6.29 g, 24.77 mmol) in dioxane (50 mL) were added potassium acetate (6.62 g, 67.56 mmol) and Pd(dppf)Cl₂ (0.82 g, 11.26 mmol). The reaction mixture was heated at 100° C. for 16 h under N₂ atmosphere. The reaction mixture was cooled to room temperature and filtered and the filter cake was washed with DCM. The organic layer was concentrated to afford the title product (5.1 g) as a yellow solid and used to the next step directly. LC-MS: [M+H]⁺=270.2.

Step 2: Methyl 3-(5-bromo-3-nitropyridin-2-yl)-1-(methyl-d3)-1H-pyrazole-5-carboxylate

To a solution of methyl 1-(methyl-d3)-3-(4,4,5,5-tetramethyl-1,3,2-dioxaborolan-2-yl)-1H-pyrazole-5-carboxylate (5 g, 18.51 mmol) and 2,5-dibromo-3-nitropyridine (4.17 g, 14.8 mmol) in THF (25 mL) and water (5 mL) were added K₂PO₄ (7.86 g, 37.03 mmol) and Pd(dppf)Cl₂ (1.35 g, 1.85 mmol). The reaction mixture was heated at 90° C. for 3 h under N₂ atmosphere. The reaction mixture was cooled to room temperature and filtered. The filter cake was washed with DCM. The organic layer was concentrated. The residue was purified by silica gel column to afford the title product (2.5 g) as a yellow solid. ¹H NMR (400 MHz, CDCl₃) δ8.84 (d, J=1.8 Hz, 1H), 8.08 (d, J=1.8 Hz, 1H), 7.34 (s, 1H), 3.91 (d, J=3.8 Hz, 3H).

Step 3: Methyl 3-(5-(1,4-dimethyl-1H-1,2,3-triazol-5-yl)-3-nitropyridin-2-yl)-1-methyl-1H-pyrazole-5-carboxylate

To a solution of methyl 3-(5-bromo-3-nitropyridin-2-yl)-1-(methyl-d3)-1H-pyrazole-5-carboxylate (5 g, 15 mmol) and Int-3 (6.5 g, 17 mmol) in DMF (50 mL) were added Pd(PPh₃)₄ (1.09 g, 0.94 mmol) and CuI (425 mg, 2.24 mmol). The reaction mixture was heated at 95° C. under N₂ atmosphere for 3 h. The reaction mixture was poured into water and extracted with DCM. The organic layer was washed with brine, dried over Na₂SO₄ and concentrated. The residue was purified by silica gel column chromatography (PE:EtOAc=5:1˜1:1˜DCM:MeOH=80:1˜40:1) to afford the title product (5 g, 96% yield) as a yellow solid. ¹H NMR (400 MHz, CDCl₃) δ8.77 (s, 1H), 7.93 (s, 1H), 7.42 (s, 1H), 4.04 (s, 3H), 3.93 (s, 3H), 2.39 (s, 3H). LC-MS: [M+H]⁺=361.1.

Step 4: Methyl 3-(5-(1,4-dimethyl-1H-1,2,3-triazol-5-yl)-3-nitropyridin-2-yl)-4-iodo-1-(methyl-d3)-1H-pyrazole-5-carboxylate

To a solution of methyl 3-(5-(1,4-dimethyl-1H-1,2,3-triazol-5-yl)-3-nitropyridin-2-yl)-1-methyl-1H-pyrazole-5-carboxylate (5 g, 13.89 mmol) in ACN (250 mL) were added Ce(NH₄)₂(NO₃)₆ (4.56 g, 8.32 mmol) and I₂ (1.76 g, 6.93 mmol) under N₂ atmosphere. The reaction mixture was heated at 80° C. under N₂ atmosphere for 2 h. Then Ce(NH₄)₂(NO₃)₆ (4.56 g, 8.31 mmol) and I₂ (1.76 g, 6.93 mmol) were added to the reaction mixture. The reaction mixture was concentrated. The residue was diluted with DCM and water, the separated aqueous phase was extracted with DCM. The organic layer was washed with brine, dried over Na₂SO₄ and concentrated to afford the title product (6.4 g, 94.6% yield) as a yellow solid. ¹H NMR (400 MHz, CDCl₃) δ 8.93 (s, 1H), 8.28 (s, 1H), 4.09 (s, 3H), 4.01 (s, 3H), 2.44 (s, 3H). LC-MS: [M+H]⁺=487.0.

Step 5: Methyl 3-(3-amino-5-(1,4-dimethyl-1H-1,2,3-triazol-5-yl)pyridin-2-yl)-4-iodo-1-(methyl-d3)-1H-pyrazole-5-carboxylate

To a solution of methyl 3-(5-(1,4-dimethyl-1H-1,2,3-triazol-5-yl)-3-nitropyridin-2-yl)-4-iodo-1-methyl-1H-pyrazole-5-carboxylate (3.2 g, 6.58 mmol) in EtOH (160 mL) and water (20 mL) were added iron powder (2.9 g, 53 mmol) and NH₄Cl (4.6 g, 79 mmol). The reaction mixture was hated at 80° C. for 1 h. The reaction mixture was filtered and the filtrate was concentrated. The residue was diluted with DCM and water, the separated aqueous phase was extracted with DCM. The organic layer was washed with brine, dried over Na₂SO₄ and concentrated. The residue was purified by silica gel column chromatography (DCM:MeOH=80:1˜60:1) to afford the title product (4.4 g, 73% yield) as a yellow solid. ¹H NMR (400 MHz, CDCl₃) δ8.09 (s, 1H), 6.99 (s, 1H), 5.16 (s, 2H), 4.01 (s, 6H), 2.37 (s, 3H). LC-MS: [M+H]⁺=457.0.

Step 6: Methyl 6-(1,4-dimethyl-1H-1,2,3-triazol-5-yl)-2-(methyl-d3)-2,4-dihydropyrazolo[3′,4′:4,5]pyrrolo[3,2-b]pyridine-3-carboxylate

To a solution of methyl 3-(3-amino-5-(1,4-dimethyl-1H-1,2,3-triazol-5-yl)pyridin-2-yl)-4-iodo-1-methyl-1H-pyrazole-5-carboxylate (100 mg, 0.18 mmol) in toluene (16 mL) were added Pd₂(dba)₃ (30 mg), Xantphos (30 mg) and Cs₂CO₃ (148 mg). The reaction mixture was hated at 160° C. by microwave for 3 h under N₂ atmosphere (26 batches). The reaction mixture was concentrated. The residue was purified by silica gel column chromatography (DCM:MeOH=80:1˜40:1) to afford the title product (535 mg, 28.8% yield) as a yellow solid. LC-MS: [M+H]⁺=329.15.

Step 7: Methyl 6-(1,4-dimethyl-1H-1,2,3-triazol-5-yl)-4-((3-fluoropyridin-2-yl)(tetrahydro-2H-pyran-4-yl)methyl)-2-(methyl-d3)-2,4- dihydropyrazolo[3′,4′:4,5]pyrrolo[3,2-b]pyridine-3-carboxylate

To a solution of methyl 6-(1,4-dimethyl-1H-1,2,3-triazol-5-yl)-2-(methyl-d3)-2,4-dihydropyrazolo[3′,4′:4,5]pyrrolo[3,2-b]pyridine-3-carboxylate (520 mg, 1.57 mmol) and (3-fluoropyridin-2-yl)(tetrahydro-2H-pyran-4-yl)methyl methanesulfonate (491 mg, 1.71 mmol) in ACN (34 mL) was added Cs₂CO₃ (595 mg, 1.8 mmol). The reaction mixture was heated at 70° C. for 12 h.The reaction mixture was concentrated. The residue was purified by prep-TLC (DCM:MeOH=20:1) to afford the title product (140 mg, 16.9% yield) as a yellow solid. LC-MS: [M+H]⁺=502.2.

### Example 14, 15 & 16

### 2-(6-(1,4-Dimethyl-1H-1,2,3-triazol-5-yl)-4-((3-fluoropyridin-2-yl)(tetrahydro-2H-pyran-4-yl)methyl)-2-(methyl-d3)-2,4-dihydropyrazolo[3′,4′:4,5]pyrrolo[3,2-b]pyridin-3-yl)propan-2-ol

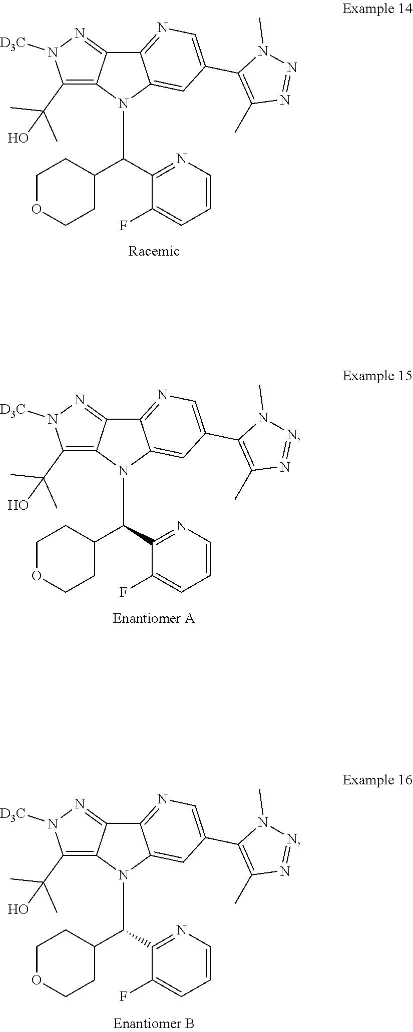 [SMILES: [2H]C([2H])([2H])n1nc2c3ncc(-c4c(C)nnn4C)cc3n(C(c3ncccc3F)C3CCOCC3)c2c1C(C)(C)O ; [2H]C([2H])([2H])n1nc2c3ncc(-c4c(C)nnn4C)cc3n([C@@H](c3ncccc3F)C3CCOCC3)c2c1C(C)(C)O ; [2H]C([2H])([2H])n1nc2c3ncc(-c4c(C)nnn4C)cc3n([C@H](c3ncccc3F)C3CCOCC3)c2c1C(C)(C)O]

Step 1: 2-(6-(1,4-Dimethyl-1H-1,2,3-triazol-5-yl)-4-((3-fluoropyridin-2-yl)(tetrahydro-2H-pyran-4-yl)methyl)-2-(methyl-d3)-2,4- dihydropyrazolo[3′,4′:4,5]pyrrolo[3,2-b]pyridin-3-yl)propan-2-ol

To a solution of methyl 6-(1,4-dimethyl-1H-1,2,3-triazol-5-yl)-4-((3-fluoropyridin-2-yl)(tetrahydro-2H-pyran-4-yl)methyl)-2-(methyl-d3)-2,4- dihydropyrazolo[3′,4′:4,5]pyrrolo[3,2-b]pyridine-3-carboxylate (140 mg, 0.28 mmol) in THF (0.8 mL) was added CH₃MgBr (14 mL, 14 mmol) at 0° C. The reaction mixture was stirred at 0° C. for 5 min. Then the reaction mixture was warmed to 66° C. and stirred at 66° C. for 40 min. The reaction mixture was cooled to −10° C. Then the mixture was quenched with sat NH₄Cl, the separated aqueous phase was extracted with DCM. The organic layer was washed with brine, dried over Na₂SO₄ and concentrated. The residue was purified by prep-TLC (DCM:MeOH=20:1) and prep-HPLC (Welch Ultimate XB-C18, 21.2*250 mm, 5 um, 20%-70% CH₃CN/water, 0.1% CF₃COOH) to afford the title product (26.3 mg, 18.8% yield) as a yellow solid. ¹H NMR (400 MHz, CDCl₃) δ 8.48 (s, 1H), 8.45 (d, J=4.4 Hz, 1H), 8.39 (s, 1H),7.42˜7.37 (m, 1H), 7.33˜7.30 (m, 1H), 7.13 (d, J=10.4 Hz, 1H), 4.01 (s, 3H), 3.99˜3.87 (m, 2H), 3.51˜3.45 (m, 1H), 3.42˜3.26 (m, 2H), 2.35 (s, 3H), 1.95 (s, 6H), 1.70˜1.46 (m, 3H), 0.92˜0.82 (m, 1H). LC-MS: [M+H]⁺=522.3. Racemic example 14 (20 mg) was separated by Chiral Prep SFC (column: Lux 5 um Cellulose-42 cm×25 cm, 5 um; Mobile phase: MeOH:EtOH=50:50; Flow rate: 25 mL/min) to give Enantiomer A example 15 (6.7 mg, 33.5% yield) and Enantiomer B example 16 (6.5 mg, 32.5% yield). Enantiomer A example 15: ¹H NMR (400 MHz, CDCl₃) δ8.43 (d, J=6.4 Hz, 1H), 8.30 (d, J=2.4 Hz, 1H), 8.00 (d, J=2 Hz, 1H), 7.40-7.33 (m, 1H), 7.30-7.28 (m, 1H), 6.99-6.95 (m, 1H), 3.98-3.83 (m, 5H), 3.52-3.43 (m, 1H), 3.44-3.23 (m, 2H), 2.31 (s, 3H), 1.91-1.89 (d, J=7.6 Hz, 6H), 1.55-1.41 (m, 3H), 0.95-0.88 (m, 1H). LC-MS: [M+H]⁺=522.3. Chiral SFC=2.819 min (Column: Lux Cellulose-4, 100*4.6 mm, 3 um H19-381245; Mobile Phase A: Ethanol; Mobile Phase B: Methanol; Flow: 1 mL/min; Detection: UV at 254 nm). Enantiomer B example 16: ¹H NMR (400 MHz, CDCl₃) δ8.43 (d, J=6.4 Hz, 1H), 8.30 (d, J=2.4 Hz, 1H), 8.00 (d, J=2 Hz, 1H), 7.40-7.33 (m, 1H), 7.30-7.28 (m, 1H), 6.99-6.95 (m, 1H), 3.98-3.83 (m, 5H), 3.52-3.43 (m, 1H), 3.44-3.23 (m, 2H), 2.31 (s, 3H), 1.91-1.89 (d, J=7.6 Hz, 6H), 1.55-1.41 (m, 3H), 0.95-0.88 (m, 1H). LC-MS: [M+H]⁺=522.3. Chiral SFC=3.328 min (Column: Lux Cellulose-4, 100*4.6 mm, 3 um H19-381245; Mobile Phase A: Ethanol; Mobile Phase B: Methanol; Flow: 1 mL/min; Detection: UV at 254 nm).

### Example 17

### Methyl 4-((3-fluoropyridin-2-yl)(tetrahydro-2H-pyran-4-yl)methyl)-2-methyl-6-(1-methyl-4-(methyl-d3)-1H-1,2,3-triazol-5-yl)-2,4- dihydropyrazolo[3′,4′:4,5]pyrrolo[3,2-b]pyridine-3-carboxylate

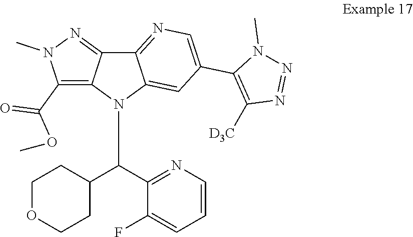 [SMILES: [2H]C([2H])([2H])c1nnn(C)c1-c1cnc2c3nn(C)c(C(=O)OC)c3n(C(c3ncccc3F)C3CCOCC3)c2c1]

Step 1: 1-Methyl-4-(methyl-d3)-5-(tributylstannyl)-1H-1,2,3-triazole

To a solution of 1-methyl-4-(methyl-d3)-1H-1,2,3-triazole (400 mg, 4 mmol) in THF (8 mL) was added n-BuLi (1.97 mL, 4.8 mmol) at −70° C. The mixture was stirred at −70° C. for 1 h. Then tributylchlorostannane (1.43 g, 4.4 mmol) was added to the reaction mixture at −70° C. The reaction mixture was stirred at −30° C. for 1 h. The reaction mixture was quenched with 1 M CeF₂ solution and sat NH₄Cl solution, the aqueous phase was extracted with EtOAc. The organic layer was washed with brine, dried over Na₂SO₄ and concentrated. The residue was purified by silica gel column chromatography (PE:EtOAc=1:0˜10:1) to afford the title product (1 g, 67% yield) as a colorless oil. ¹H NMR (400 MHz, CDCl₃) δ4.03 (s, 3H), 1.61-1.43 (m, 6H), 1.34 (dt, J=14.5, 7.2 Hz, 6H), 1.26-1.15 (m, 6H), 0.90 (t, J=7.3 Hz, 9H). LC-MS: [M+H]⁺=391.1.

Step 2: Methyl 1-methyl-3-(5-(1-methyl-4-(methyl-d3)-1H-1,2,3-triazol-5-yl)-3-nitropyridin-2-yl)-1H-pyrazole-5-carboxylate

To a solution of methyl 3-(5-bromo-3-nitropyridin-2-yl)-1-methyl-1H-pyrazole-5-carboxylate (3 g, 8.82 mmol) and 1-methyl-4-(methyl-d3)-1H-1,2,3-triazole (3.3 g, 8.46 mmol) in DMF (50 mL) were added Pd(PPh₃)₄ (656 mg, 0.57 mmol) and CuI (249 mg, 1.15 mmol) under N₂ atmosphere. The mixture was heated at 95° C. for 2 h under N₂ atmosphere. The reaction mixture was poured into water and extracted with DCM. The organic layer was washed with brine, dried over Na₂SO₄ and concentrated. The residue was purified by silica gel column chromatography (PE:DCM=2:1˜DCM:MeOH=100:1) to afford the title product (2.5 g, 79.6% yield) as a yellow solid. ¹H NMR (400 MHz, CDCl₃) δ8.78 (d, J=1.9 Hz, 1H), 7.91 (d, J=1.9 Hz, 1H), 7.43 (s, 1H), 4.23 (s, 3H), 4.06 (s, 3H), 3.94 (s, 3H). LC-MS: [M+H]⁺=361.1.

Step 3: Methyl 4-iodo-1-methyl-3-(5-(1-methyl-4-(methyl-d3)-1H-1,2,3-triazol-5-yl)-3-nitropyridin-2-yl)-1H-pyrazole-5-carboxylate

To solution of methyl 1-methyl-3-(5-(1-methyl-4-(methyl-d3)-1H-1,2,3-triazol-5-yl)-3-nitropyridin-2-yl)-1H-pyrazole-5-carboxylate (2.5 g, 6.94 mmol) in ACN (120 mL) were added Ce(NH₄)₂(NO₃)₆ (2.1 g, 4.17 mmol) and I₂ (811 mg, 3.47 mmol) under N₂ atmosphere. The reaction mixture was heated at 80° C. for 2 h. Then Ce(NH₄)₂(NO₃)₆ (2.1 g, 4.17 mmol), I₂ (811 mg, 3.47 mmol) was added to the reaction mixture under N₂ atmosphere. The reaction mixture was concentrated in vacuum. The residue was diluted with DCM and water, the separated aqueous phase was extracted with DCM. The organic layer was washed with brine, dried over Na₂SO₄ and concentrated to afford the title product (3.1 g, 91.7% yield) as a yellow solid. ¹H NMR (400 MHz, CDCl₃) δ8.93 (d, J=1.9 Hz, 1H), 8.28 (d, J=1.9 Hz, 1H), 4.29 (s, 3H), 4.10 (s, 3H), 4.01 (s, 3H). LC-MS: [M+H]⁺=487.0.

Step 4: Methyl 3-(3-amino-5-(1-methyl-4-(methyl-d3)-1H-1,2,3-triazol-5-yl)pyridin-2-yl)-4-iodo-1-methyl-1H-pyrazole-5-carboxylate

To a solution of methyl 4-iodo-1-methyl-3-(5-(1-methyl-4-(methyl-d3)-1H-1,2,3-triazol-5-yl)-3-nitropyridin-2-yl)-1H-pyrazole-5-carboxylate (3.1 g, 6.38 mmol) in EtOH (160 mL) and water (20 mL) were added iron powder (2.8 g, 51 mmol) and NH₄Cl (4.1 g, 77 mmol). The reaction mixture was hated at 80° C. for 1 h. The reaction mixture was filtered and the filtrate was concentrated in vacuum. The residue was diluted with DCM and water, the separated aqueous phase was extracted with DCM. The organic layer was washed with brine, dried over Na₂SO₄ and concentrated. The residue was purified by silica gel column chromatography (PE:EtOAc=1:1˜DCM:MeOH=50:1) to afford the title product (2.0 g, 69% yield) as a yellow solid. ¹H NMR (400 MHz, CDCl₃) δ8.08 (d, J=1.5 Hz, 1H), 6.99 (d, J=1.7 Hz, 1H), 5.17 (s, 2H), 4.27 (s, 3H), 4.01 (s, 6H). LC-MS: [M+H]⁺=457.0.

Step 5: Methyl 2-methyl-6-(1-methyl-4-(methyl-d3)-1H-1,2,3-triazol-5-yl)-2,4-dihydropyrazolo[3′,4′:4,5]pyrrolo[3,2-b]pyridine-3- carboxylate

To a solution of methyl 3-(3-amino-5-(1-methyl-4-(methyl-d3)-1H-1,2,3-triazol-5-yl)pyridin-2-yl)-4-iodo-1-methyl-1H-pyrazole-5-carboxylate (100 mg, 0.18 mmol) in toluene (16 mL) were added Pd₂(dba)₃ (30 mg), Xantphos (30 mg) and Cs₂CO₃ (148 mg). The reaction mixture was heated at 160° C. by microwave for 3 h under N₂ atmosphere (20 batches). The reaction mixture was concentrated in vacuum. The residue was purified by silica gel column chromatography (DCM:MeOH=80:1˜40:1) to afford the title product (492 mg, 34.4% yield) as a yellow solid. LC-MS: [M+H]⁺=329.2.

Step 6: Methyl 4-((3-fluoropyridin-2-yl)(tetrahydro-2H-pyran-4-yl)methyl)-2-methyl-6-(1-methyl-4-(methyl-d3)-1H-1,2,3-triazol-5-yl)-2,4-dihydropyrazolo[3′,4′:4,5]pyrrolo[3,2-b]pyridine-3-carboxylate

To a solution of methyl 2-methyl-6-(1-methyl-4-(methyl-d3)-1H-1,2,3-triazol-5-yl)-2,4-dihydropyrazolo[3′,4′:4,5]pyrrolo[3,2-b]pyridine-3- carboxylate (492 mg, 1.5 mmol) and (3-fluoropyridin-2-yl)(tetrahydro-2H-pyran-4-yl)methyl methanesulfonate (441 mg, 1.6 mmol) in CAN (2 mL) was added Cs₂CO₃ (693 mg, 1.8 mmol) at 20° C. The reaction mixture was heated at 70° C. for 12 h. The reaction was concentrated. The residue was purified by prep-TLC (DCM:MeOH=20:1) to afford the title product (125 mg, 15.9% yield) as a yellow solid. ¹H NMR (400 MHz, CDCl₃) δ8.45 (d, J=4.4 Hz, 1H), 8.38 (s, 1H), 8.22 (s, 1H), 7.38˜7.31 (m, 2H), 6.88 (d, J=11.2 Hz, 1H), 4.48 (s, 3H), 4.11 (s, 3H), 4.00˜3.88 (m, 4H), 3.87˜3.81 (m, 1H), 3.54˜3.51 (m, 1H), 3.38˜3.31 (m, 2H), 1.75˜1.72 (m, 1H), 1.55˜1.50 (m, 2H), 1.20˜1.88 (m, 1H). LC-MS: [M+H]⁺=522.2.

### Example 18, 19 & 20

### 2-(4-((3-Fluoropyridin-2-yl)(tetrahydro-2H-pyran-4-yl)methyl)-2-methyl-6-(1-methyl-4-(methyl-d3)-1H-1,2,3-triazol-5-yl)-2,4- dihydropyrazolo[3′,4′:4,5]pyrrolo[3,2-b]pyridin-3-yl)propan-2-ol

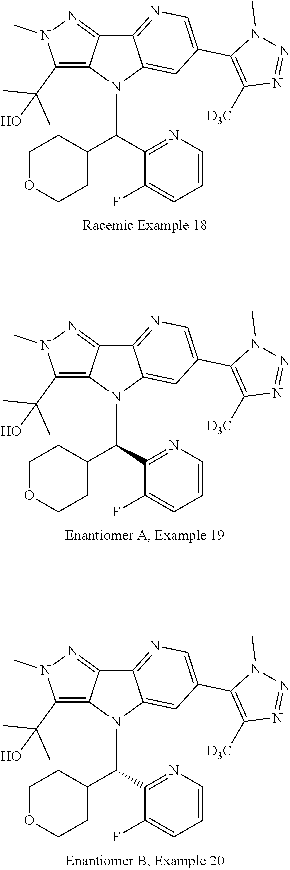 [SMILES: [2H]C([2H])([2H])c1nnn(C)c1-c1cnc2c3nn(C)c(C(C)(C)O)c3n(C(c3ncccc3F)C3CCOCC3)c2c1 ; [2H]C([2H])([2H])c1nnn(C)c1-c1cnc2c3nn(C)c(C(C)(C)O)c3n([C@@H](c3ncccc3F)C3CCOCC3)c2c1 ; [2H]C([2H])([2H])c1nnn(C)c1-c1cnc2c3nn(C)c(C(C)(C)O)c3n([C@H](c3ncccc3F)C3CCOCC3)c2c1]

Step 1: 2-(4-((3-Fluoropyridin-2-yl)(tetrahydro-2H-pyran-4-yl)methyl)-2-methyl-6-(1-methyl-4-(methyl-d3)-1H-1,2,3-triazol-5-yl)-2,4- dihydropyrazolo[3′,4′:4,5]pyrrolo[3,2-b]pyridin-3-yl)propan-2-ol

To a solution of methyl 4-((3-fluoropyridin-2-yl)(tetrahydro-2H-pyran-4-yl)methyl)-2-methyl-6-(1-methyl-4-(methyl-d3)-1H-1,2,3-triazol-5-yl)-2,4-dihydropyrazolo[3′,4′:4,5]pyrrolo[3,2-b]pyridine-3-carboxylate (125 mg, 0.24 mmol) in THF (0.8 mL) was added CH₃MgCl (12 mL, 12 mmol) at 0° C. The reaction mixture was stirred at 0° C. for 5 min. Then the reaction mixture was warmed to 66° C. and stirred at 66° C. for 40 min. The reaction mixture was cooled to −10° C. The mixture was quenched with sat NH₄Cl and extracted with DCM. The organic layer was washed with brine, dried over Na₂SO₄ and concentrated. The residue was purified by prep-TLC (DCM:MeOH=20:1) and prep-HPLC (Welch Ultimate XB-C18, 21.2*250 mm, 5 um, 20%-70% CH₃CN/water, 0.1% CF₃COOH) to afford the title product (37.5 mg, 30% yield) as a yellow solid. ¹H NMR (400 MHz, CDCl₃) δ8.43 (s, 2H), 8.26 (s, 1H), 7.40 (t, J=8.0 Hz, 1H), 7.32˜7.30 (m, 1H), 7.09 (d, J=10.8 Hz, 1H), 4.27 (s, 3H), 3.99 (s,3H), 3.95˜3.85 (m, 2H), 3.50˜3.44 (m, 1H), 3.30˜3.25 (m 2H), 1.91 (s, 6H), 1.71˜1.41 (m, 3H), 0.92˜0.88 (m, 1H). LC-MS: [M+H]⁺=522.3. Racemic example 18 (34.3 mg) was separated by Chiral Prep SFC (column: Lux Sum Cellulose-42 cm×25 cm, 5 um; Mobile phase: MeOH:EtOH=50:50; Flow rate: 25 mL/min) to give Enantiomer A example 19 (11.8 mg, 34.4% yield) and Enantiomer B example 20 (11.6 mg, 33.8% yield). Enantiomer A example 19: ¹H NMR (400 MHz, CDCl₃) δ8.44-8.42 (m, 1H), 8.29 (d, J=2.4 Hz, 1H), 7.98 (d, J=2.4 Hz, 1H), 7.40-7.33 (m, 1H), 7.30-7.28 (m, 1H), 6.99-6.95 (m, 1H), 4.24 (s, 3H), 3.98-3.91 (m, 4H), 3.90-3.82 (m, 1H), 3.52-3.43 (m, 1H), 3.37-3.23 (m, 2H), 1.90 (d, J=8.4 Hz, 6H), 1.58-1.40 (m, 3H), 0.97-0.88 (m, 1H). LC-MS: [M+H]⁺=522.3. Chiral SFC=2.985 min (Column: Lux Cellulose-4, 100*4.6 mm, 3 um H19-381245; Mobile Phase A: Ethanol; Mobile Phase B: Methanol; Flow: 1 mL/min; Detection: UV at 254 nm). Enantiomer B example 20: ¹H NMR (400 MHz, CDCl₃) δ8.44-8.42 (m, 1H), 8.29 (d, J=2.4 Hz, 1H), 7.98 (d, J=2.4 Hz, 1H), 7.40-7.33 (m, 1H), 7.30-7.28 (m, 1H), 6.99-6.95 (m, 1H), 4.24 (s, 3H), 3.98-3.91 (m, 4H), 3.90-3.82 (m, 1H), 3.52-3.43 (m, 1H), 3.37-3.23 (m, 2H), 1.90 (d, J=8.4 Hz, 6H), 1.58-1.40 (m, 3H), 0.97-0.88 (m, 1H). LC-MS: [M+H]⁺=522.3. Chiral SFC=3.527 min (Column: Lux Cellulose-4, 100*4.6 mm, 3 um H19-381245; Mobile Phase A: Ethanol; Mobile Phase B: Methanol; Flow: 1 mL/min; Detection: UV at 254 nm).

### Example 21

Methyl 4-((3-fluoropyridin-2-yl)(tetrahydro-2H-pyran-4-yl)methyl)-2-(methyl-d3)-6-(1-methyl-4-(methyl-d3)-1H-1,2,3-triazol-5-yl)-2,4- dihydropyrazolo[3′,4′:4,5]pyrrolo[3,2-b]pyridine-3-carboxylate

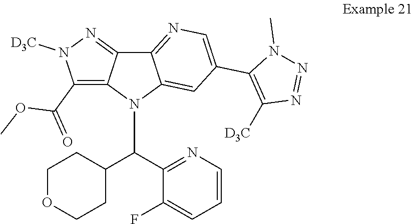 [SMILES: [2H]C([2H])([2H])c1nnn(C)c1-c1cnc2c3nn(C([2H])([2H])[2H])c(C(=O)OC)c3n(C(c3ncccc3F)C3CCOCC3)c2c1]

Step 1: Methyl 1-(methyl-d3)-3-(5-(1-methyl-4-(methyl-d3)-1H-1,2,3-triazol-5-yl)-3-nitropyridin-2-yl)-1H-pyrazole-5-carboxylate

To a solution of methyl 3-(5-bromo-3-nitropyridin-2-yl)-1-(methyl-d3)-1H-pyrazole-5-carboxylate (3 g, 8.75 mmol) and 1-methyl-4-(methyl-d3)-5-(tributylstannyl)-1H-1,2,3-triazole (3.4 g, 8.72 mmol) in DMF (45 mL) were added Pd(PPh₃)₄ (656 mg, 0.49 mmol) and CuI (249 mg, 1.31 mmol) under N₂ atmosphere. The mixture was heated at 95° C. for 2 h under N₂ atmosphere. The reaction mixture was poured into water and extracted with DCM. The organic layer was washed with brine, dried over Na₂SO₄ and concentrated. The residue was purified by silica gel column chromatography (PE:DCM=2:1˜DCM:MeOH=100:1) to afford the title product (2.9 g, 89% yield) as a yellow solid. ¹H NMR (400 MHz, CDCl₃) δ8.78 (d, J=1.9 Hz, 1H), 7.91 (d, J=1.9 Hz, 1H), 7.43 (s, 1H), 4.06 (s, 3H), 3.942 (s, 3H). LC-MS: [M+H]⁺=364.2.

Step 2: Methyl 4-iodo-1-(methyl-d3)-3-(5-(1-methyl-4-(methyl-d3)-1H-1,2,3-triazol-5-yl)-3-nitropyridin-2-yl)-1H-pyrazole-5- carboxylate

To solution of methyl 1-(methyl-d3)-3-(5-(1-methyl-4-(methyl-d3)-1H-1,2,3-triazol-5-yl)-3-nitropyridin-2-yl)-1H-pyrazole-5-carboxylate (2.9 g, 7.99 mmol) in ACN (150 mL) were added Ce(NH₄)₂(NO₃)₆ (2.66 g, 4.85 mmol) and I₂ (1.02 g, 4.05 mmol) under N₂ atmosphere. The reaction mixture was heated at 80° C. for 2 h. Then Ce(NH₄)₂(NO₃)₆ (2.66 g, 4.854 mmol) and I₂ (1.02 g, 4.045 mmol) were added under N₂ atmosphere. The reaction mixture was concentrated. The residue was diluted with DCM and water, the separated aqueous phase was extracted with DCM). The organic layer was washed with brine and dried over Na₂SO₄ and concentrated to afford the title product (3.3 g, 87% yield) as a yellow solid. ¹H NMR (400 MHz, CDCl₃) δ8.93 (d, J=1.3 Hz, 1H), 8.28 (d, J=1.5 Hz, 1H), 4.10 (s, 3H), 4.01 (s, 3H). LC-MS: [M+H]⁺=490.0.

Step 3: Methyl 3-(3-amino-5-(1-methyl-4-(methyl-d3)-1H-1,2,3-triazol-5-yl)pyridin-2-yl)-4-iodo-1-(methyl-d3)-1H-pyrazole-5- carboxylate

To a solution of methyl 4-iodo-1-(methyl-d3)-3-(5-(1-methyl-4-(methyl-d3)-1H-1,2,3-triazol-5-yl)-3-nitropyridin-2-yl)-1H-pyrazole-5- carboxylate (3.3 g, 6.75 mmol) in EtOH (160 mL) and water (20 mL) were added iron powder (2.8 g, 50 mmol) and NH₄Cl (4.1 g, 76 mmol). The reaction mixture was heated at 80° C. for 1 h. The reaction mixture was filtered and the filtrate was concentrated. The residue was diluted with DCM and water, the separated aqueous phase was extracted with DCM. The organic layer was washed with brine, dried over Na₂SO₄ and concentrated. The residue was purified by silica gel column chromatography (PE:EtOAc=1:1˜DCM:MeOH=50:1) to afford the title product (2.6 g, 84% yield) as a yellow solid. ¹H NMR (400 MHz, CDCl₃) δ8.08 (d, J=1.8 Hz, 1H), 6.99 (d, J=1.8 Hz, 1H), 5.15 (s, 2H), 4.07 (d, J=1.2 Hz, 6H). LC-MS: [M+H]⁺=460.1.

Step 4: Methyl 2-(methyl-d3)-6-(1-methyl-4-(methyl-d3)-1H-1,2,3-triazol-5-yl)-2,4-dihydropyrazolo[3′,4′:4,5]pyrrolo[3,2-b]pyridine-3-carboxylate

To a solution of methyl 3-(3-amino-5-(1-methyl-4-(methyl-d3)-1H-1,2,3-triazol-5-yl)pyridin-2-yl)-4-iodo-1-(methyl-d3)-1H-pyrazole-5- carboxylate (100 mg, 0.18 mmol) in toluene (16 mL) were added Pd₂(dba)₃ (30 mg, 0.033 mmol), Xantphos (30 mg, 0.052 mmol) and Cs₂CO₃ (148 mg, 0.46 mmol). The reaction solution was heated at 160° C. by microwave for 3 h under N₂ atmosphere (26 batches). The reaction mixture was concentrated in vacuum. The residue was purified by silica gel column chromatography (DCM:MeOH=80:1˜40:1) to afford the product (600 mg, 32.2% yield) as a yellow solid. LC-MS: [M+H]⁺=332.1.

Step 5: 1-(4-((3-Fluoropyridin-2-yl)(tetrahydro-2H-pyran-4-yl)methyl)-2-(methyl-d3)-6-(1-methyl-4-(methyl-d3)-1H-1,2,3-triazol-5-yl)-2,4-dihydropyrazolo[3′,4′:4,5]pyrrolo[3,2-b]pyridin-3-yl)ethan-1-one

To a solution of methyl 2-(methyl-d3)-6-(1-methyl-4-(methyl-d3)-1H-1,2,3-triazol-5-yl)-2,4-dihydropyrazolo[3′,4′:4,5]pyrrolo[3,2-b]pyridine- 3-carboxylate (600 mg, 1.81 mmol) and (3-fluoropyridin-2-yl)(tetrahydro-2H-pyran-4-yl)methyl methanesulfonate (576 mg, 1.99 mmol) in ACN (48 mL) was added Cs₂CO₃ (792 mg, 2.44 mmol) at 20° C. The reaction mixture was heated at 70° C. for 12 h. The reaction mixture was concentrated. The residue was purified by prep-TLC (DCM:MeOH=20:1) to afford the product (190 mg, 20% yield) as a yellow solid. LC-MS: [M+H]⁺=525.3.

### Example 22, 23 & 24

### 2-(4-((3-Fluoropyridin-2-yl)(tetrahydro-2H-pyran-4-yl)methyl)-2-(methyl-d3)-6-(1-methyl-4-(methyl-d3)-1H-1,2,3-triazol-5-yl)-2,4- dihydropyrazolo[3′,4′:4,5]pyrrolo[3,2-b]pyridin-3-yl)propan-2-ol

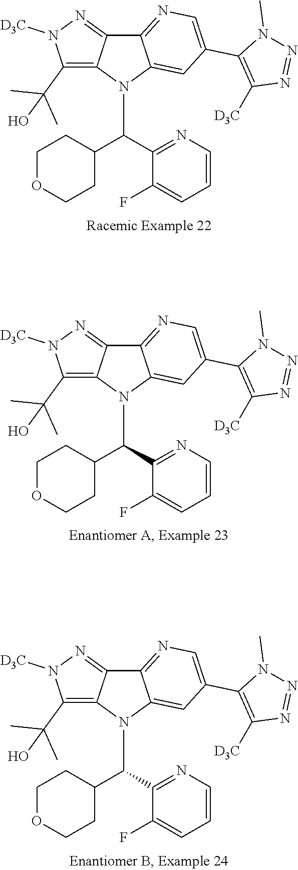 [SMILES: [2H]C([2H])([2H])c1nnn(C)c1-c1cnc2c3nn(C([2H])([2H])[2H])c(C(C)(C)O)c3n(C(c3ncccc3F)C3CCOCC3)c2c1 ; [2H]C([2H])([2H])c1nnn(C)c1-c1cnc2c3nn(C([2H])([2H])[2H])c(C(C)(C)O)c3n([C@@H](c3ncccc3F)C3CCOCC3)c2c1 ; [2H]C([2H])([2H])c1nnn(C)c1-c1cnc2c3nn(C([2H])([2H])[2H])c(C(C)(C)O)c3n([C@H](c3ncccc3F)C3CCOCC3)c2c1]

Step 1: 2-(4-((3-Fluoropyridin-2-yl)(tetrahydro-2H-pyran-4-yl)methyl)-2-(methyl-d3)-6-(1-methyl-4-(methyl-d3)-1H-1,2,3-triazol-5-yl)-2,4-dihydropyrazolo[3′,4′:4,5]pyrrolo[3,2-b]pyridin-3-yl)propan-2-ol

To a solution of 1-(4-((3-fluoropyridin-2-yl)(tetrahydro-2H-pyran-4-yl)methyl)-2-(methyl-d3)-6-(1-methyl-4-(methyl-d3)-1H-1,2,3-triazol-5-yl)-2,4-dihydropyrazolo[3′,4′:4,5]pyrrolo[3,2-b]pyridin-3-yl)ethan-1-one (190 mg, 0.36 mmol) in THF (2.8 mL) was added MeMgCl (19 mL, 19 mmol) at 0° C. The reaction mixture was stirred at 0° C. for 5 min. Then the reaction mixture was warmed to 66° C. and stirred at 66° C. for 40 min. The reaction mixture was cooled to −10° C. Then the mixture was quenched with sat NH₄Cl, the separated aqueous phase was extracted with DCM. The organic layer was washed with brine, dried over Na₂SO₄ and concentrated. The residue was purified by prep-TLC (DCM:MeOH=20:1) and prep-HPLC (Welch Ultimate XB-C18, 21.2*250 mm, 5 um, 30%-80% CH₃CN/water, 0.1% CF₃COOH) to afford the title product (52 mg, 27.4% yield) as a yellow solid. ¹H NMR (400 MHz, CDCl₃) δ8.44 (s, 1H), 8.45 (d, J=4.4 Hz, 1H), 8.37 (s, 1H), 7.42 (t, J=8.4 Hz, 1H), 7.33˜7.29 (m, 1H), 7.13 (d, J=10.8 Hz, 1H), 4.01 (s, 3H), 3.99˜3.87 (m, 2H), 3.51˜3.45 (m, 1H), 3.31˜3.26 (m, 2H), 1.91 (s, 6H), 1.70˜1.42 (m, 3H), 0.92˜088 (m, 1H). LC-MS: c[M+H]⁺=525.3. Racemic example 22 (39.6 mg) was separated by Chiral Prep SFC (column: Lux Sum Cellulose-42 cm×25 cm, 5 um; Mobile phase: MeOH:EtOH=50:50; Flow rate: 25 mL/min) to give Enantiomer A example 23 (13.9 mg, 35.1% yield) and Enantiomer B example 24 (15.1 mg, 38.1% yield). Enantiomer A example 17: ¹H NMR (400 MHz, CDCl₃) δ8.44-8.42 (m, 1H), 8.29 (d, J=2 Hz, 1H), 7.98 (d, J=2.4 Hz, 1H), 7.39-7.33 (m, 1H), 7.30-7.27 (m, 1H), 6.96 (d, J=13.6 Hz, 1H), 3.99-3.83 (m, 5H), 3.52-3.44 (m, 1H), 3.36-3.23 (m, 2H), 1.90 (d, J=13.6 Hz, 6H), 1.54-1.37 (m, 3H), 0.96-0.88 (m, 1H). LC-MS: [M+H]⁺=525.3. Chiral SFC=2.917 min (Column: Lux Cellulose-4, 100*4.6 mm, 3 um H19-381245; Mobile Phase A: Ethanol; Mobile Phase B: Methanol; Flow: 1 mL/min; Detection: UV at 254 nm). Enantiomer B example 18: ¹H NMR (400 MHz, CDCl₃) δ8.44-8.42 (m, 1H), 8.29 (d, J=2 Hz, 1H), 7.98 (d, J=2.4 Hz, 1H), 7.39-7.33 (m, 1H), 7.30-7.27 (m, 1H), 6.96 (d, J=13.6 Hz, 1H), 3.99-3.83 (m, 5H), 3.52-3.44 (m, 1H), 3.36-3.23 (m, 2H), 1.90 (d, J=13.6 Hz, 6H), 1.54-1.37 (m, 3H), 0.96-0.88 (m, 1H). LC-MS: [M+H]⁺=525.3. Chiral SFC=3.450 min (Column: Lux Cellulose-4, 100*4.6 mm, 3 um H19-381245; Mobile Phase A: Ethanol; Mobile Phase B: Methanol; Flow: 1 mL/min; Detection: UV at 254 nm).

### Pharmacological Testing

### 1. BRD4 (BD1) Binding Assay

The BRD4 (BD1) biochemical binding assay was carried out by Sundia MediTech Co., Ltd.
Materials and suppliers:
BRD4(D1): Reaction Biology Company; catalog #: RD-11-157; batch #1319
Positive control compound: AZD5153; Selleck, catalog #, S8344, batch #, 1
Peptide: GL China; catalog #329934; batch #, P171229-JQ329934
DMSO: Sigma; catalog #, D8418-1L; batch #, SHBG3288V
OptiPlate-384: PerkinElmer; catalog #6007270, batch #, 8240-17211
Instruments:
Centrifuge (manufacturer: Eppendorf, model: 5430)
Microplate reader (manufacturer: Perkin Elmer, model: EnVision)
Sonic pipetting system (manufacturer: Labcyte, model: Echo® 550)
In the assay, the HTRF method uses an anti-GST antibody with a cryptate chelate-labeled europium element as the donor, and the d2 or XL665-labeled streptavidin (having high affinity with biotin) as the acceptor to study the interaction between BRD4 (D1) with GST tag and biotin-labeled acetylated peptide. When the donor and acceptor are close to each other due to the binding of BRD4 (D1) to the biotin-labeled peptide, the excitation of the donor triggers fluorescence resonance energy transfer (FRET) to the acceptor, which then emits fluorescence at a specific wavelength (665 nm).
General assay procedure: 20 nL of compound solution was transferred to the 384-well plate by Labcyte Echo® 550 liquid handler, and 5 μL of BRD4 (BD1) solution from Reaction Biology Company, RD-11-157, or the assay buffer was added to each well. After centrifuge for 1 minute and incubating 15 minutes at rt, the 5 μL peptide (GL China, Catalog #329934, Batch, P171229-JQ329934) was added to each well. After centrifuge for 1 minute at 1000 rpm, 10 μL detect solution was added to each well. After incubating for 60 minutes, centrifuging 1 minute, the signals were measured by Microplate reader. Data analysis: The inhibition percentage in the presence of the compound was calculated based on the following formula:

% Inhibition=(Signal_max−Signal sample/Signal_max−Signal_min)×100

Fitting the data in GrphaPad Prism V5.0 software with log(inhibitor) vs. response-Variable slope, the IC50 was obtained.

The results of BRD4(BD1) binding assay are in the following Table 1.

TABLE 1
The result of BRD4(BD1) binding assay

| | BRD4(BD1) |
|---|---|
| Example # | IC50/nM |
| Example 2 | 0.3 |
| Example 3 | 1.8 |
| Example 4 | 0.18 |
| Example 6 | 0.61 |
| Example 7 | 8.4 |
| Example 8 | 0.18 |
| Example 9 | 0.51 |
| Example 10 | 0.61 |
| Example 11 | 6.8 |
| Example 12 | 0.19 |
| Example 14 | 0.53 |
| Example 15 | 4.8 |
| Example 16 | 0.18 |
| Example 17 | 0.59 |
| Example 18 | 0.52 |
| Example 19 | 5.0 |
| Example 20 | 0.17 |
| Example 22 | 0.34 |
| Example 23 | 4.4 |
| Example 24 | 0.17 |

### 2. Cell Proliferation Assay

Materials:

Cell Line

| Cell Line | Culture | Properties | Source Culture Medium |
|---|---|---|---|
| MV-4-11 | Suspension | ATCC | IMDM + 10% FBS |

Reagents

| Reagents | Vender | Catalog # |
|---|---|---|
| CellTiter-Glo ® | Promega | G7571 IMDM |
| IMDM | Invitrogen | 12440061 |
| Fetal bovine serum | EXCELL | FND500 |
| 0.25% Trypsin-EDTA | Gibco | 25200-072 |
| Dimethyl sulfoxide | Sigma-Aldrich | D2650 |

Instruments and software

| Name | Vendor | Model |
|---|---|---|
| Incubator | Thermo | 3111 |
| Biological safety cabinet | NuAire | NU-543-600S |
| Inverted microscope | Nikon | TS-100 |
| Automated cell counter | Life technologies | Countess II |
| Multilabel Reader | PerkinElmer | EnVision |
| GraphPad Prism 5.0 | / | / |
| software | | |

In order to evaluate the activity of the compound on cells, the Cell Titer-Glo assay was used for detection. The cells were incubated in a 37° C. incubator and treated with compounds for 72 hours. At the end of the reaction, the number of living cells in the culture is measured by quantitative determination of ATP. ATP is an indicator of the metabolism of living cells. With Cell Titer-Glo, the luminescence signal produced by cell lysis is proportional to the amount of ATP present, and the amount of ATP is directly proportional to the number of cells in the culture. These signal ratios reflect the cell activity of the compound at that concentration in the well under this condition.
(1) Seeding cells
Trypsinize cells and count cell density with the automated cell counter.
Dilute cell suspension to required density according to seeding density.
Seed 100 ul cells into 96-well plate in growth medium according to the plate map. Medium only is used as background control (Min).
Incubate at 37° C., 5% CO₂ overnight.
(2) Compound treatment
Make 200× compound solution in DMSO.
Dilute compounds with growth medium to 3× final concentration by addition of 3 ul 200× compounds to 197 ul growth medium.
Add 50 ul diluted compounds to cells and incubate at 37° C., 5% CO₂ for 72 h.
(3) Measurement
Equilibrate the assay plate to room temperature prior to measurement.
Add 40 ul of CellTiter-Glo® Reagent into each well.
Mix contents for 2 minutes on an orbital shaker to induce cell lysis.
Incubate at room temperature for 60 minutes to stabilize luminescent signal.
Record luminescence on Envision.
(4) Data analysis
(1) Using GraphPad Prism 5.
(2) % Inh=(Max signal−Compound signal)/(Max signal−Min signal)×100.
(3) Max signal was obtained from the action of DMSO.
(4) Min signal was obtained from the action of medium only.

The results of the cellular proliferation activity are in the following Table 2.

TABLE 2
The result of the cellular proliferation activity

| | MV-4-11 |
|---|---|
| Example # | IC50/nM |
| Example 2 | 1.48 |
| Example 3 | 36.87 |
| Example 4 | 1.28 |
| Example 6 | 1.98 |
| Example 7 | 139.4 |
| Example 8 | 0.75 |
| Example 9 | 1.52 |
| Example 10 | 3.42 |
| Example 11 | 63.32 |
| Example 12 | 0.52 |
| Example 14 | 1.77 |
| Example 15 | 72.17 |
| Example 16 | 0.49 |
| Example 17 | 3.30 |
| Example 18 | 1.94 |
| Example 19 | 85.28 |
| Example 20 | 0.81 |
| Example 22 | 1.40 |
| Example 23 | 66.75 |
| Example 24 | 0.47 |

1. A compound of the formula I, a pharmaceutically acceptable salt thereof or stereoisomer thereof: 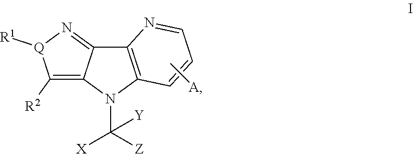
wherein:
Q is selected form N, O and S, provided that when Q is O or S, R¹ is absent;
A is selected from the following: 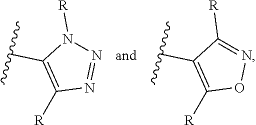
each of R is independently selected from hydrogen, optionally substituted (C₁-C₆)alkyl, halogen, and —CD₃;
X and Y are independently selected from phenyl; 6-membered heteroaryl containing 1 or 2 heteroatoms selected from N; 6-membered heterocyclic containing 1 or 2 heteroatoms selected from O, S; and 6-membered carbocyclic; and each of which at each occurrence is independently optionally substituted with —C₁₋₃alkyl or halogen;
Z is selected from hydrogen, —F, —Cl, —OH, —C₁₋₃alkyl and —C₁₋₃alkoxy;
R¹ is selected from halogen, optionally substituted (C₁-C₆)alkyl, optionally substituted (C₂-C₆)alkenyl, and optionally substituted (C₂-C₆)alkynyl; and
R² is selected from —COOR²¹ and —(CH₂)ₙ—CR²²R²³—OH, wherein R²¹ is hydrogen, or optionally substituted (C₁-C₆)alkyl, each of R²² and R²³ is selected from hydrogen, halogen, and —C₁₋₆alkyl; n is selected from 0, 1, 2, 3, 4, 5 and 6.

2. The compound of formula I, a pharmaceutically acceptable salt thereof or stereoisomer thereof according to claim 1, wherein the compound is of formula I-1: 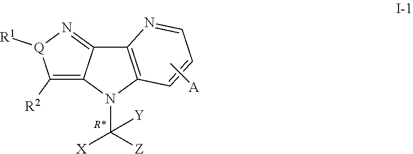
R* in the formula I-1 indicates that the absolute configuration of the carbon that is substituted with the X, Y and Z is R configuration when the carbon is a chiral carbon.

3. The compound of formula I, a pharmaceutically acceptable salt thereof or stereoisomer thereof according to claim 1, wherein the compound is of formula 1-2: 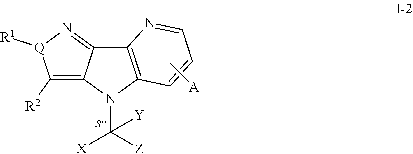
S* in the formula 1-2 indicates that the absolute configuration of the carbon that is substituted with the X, Y and Z is S configuration when the carbon is a chiral carbon.

4. The compound of formula I, a pharmaceutically acceptable salt thereof or stereoisomer thereof according to claim 1, wherein A is selected from the following:

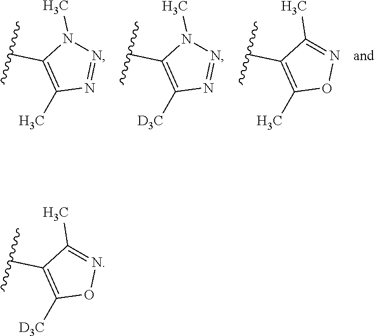

5. The compound of formula I, a pharmaceutically acceptable salt thereof or stereoisomer thereof according to claim 1, wherein A is selected from the following:

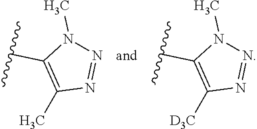

6. The compound of formula I, a pharmaceutically acceptable salt thereof or stereoisomer thereof according to claim 1, wherein Q is N.

7. The compound of formula I, a pharmaceutically acceptable salt thereof or stereoisomer thereof according to claim 1, wherein X and Y are independently selected from phenyl;

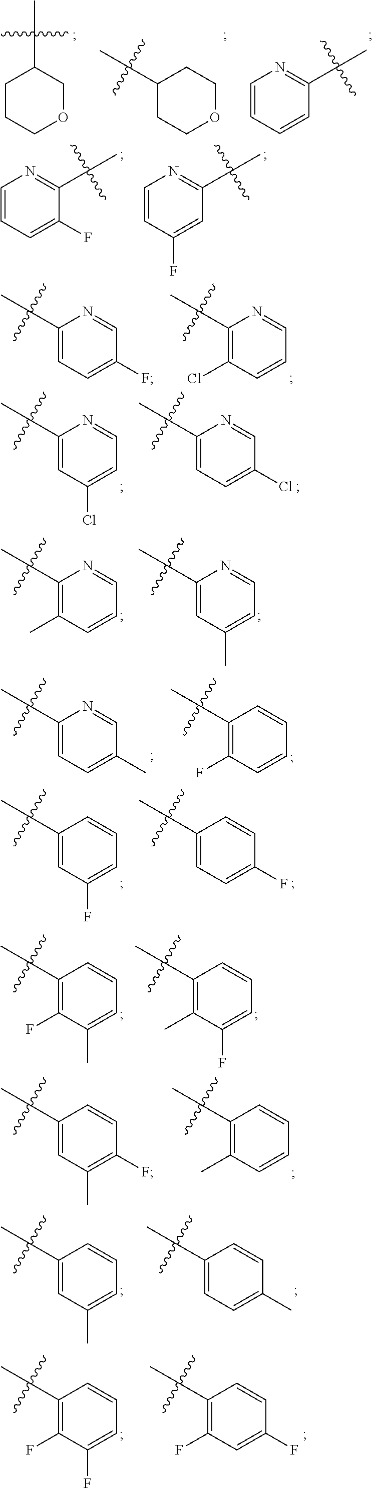

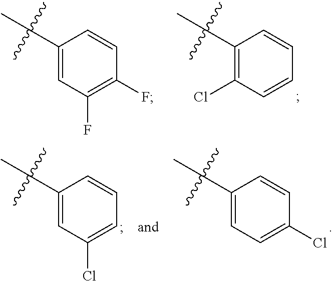

8. The compound of formula I, a pharmaceutically acceptable salt thereof or stereoisomer thereof according to claim 1, wherein X and Y are independently selected from phenyl;

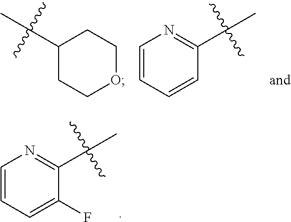

9. The compound of formula I, a pharmaceutically acceptable salt thereof or stereoisomer thereof according to claim 1, wherein Z is selected from hydrogen, —F, —Cl, —OH, methyl, ethyl, propyl, isopropyl, methoxy, ethoxy, propoxy and isopropoxy.

10. The compound of formula I, a pharmaceutically acceptable salt thereof or stereoisomer thereof according to claim 1, wherein Z is hydrogen or methyl.

11. The compound of formula I, a pharmaceutically acceptable salt thereof or stereoisomer thereof according to claim 1, wherein 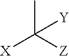
is selected from 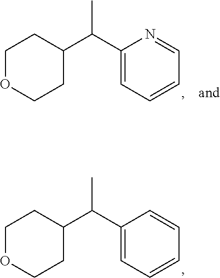
in which the pyridine ring and benzene ring are each independently optionally substituted with 1, 2 or 3 substituents, and said each of substituents at each occurrence is selected from —F, —Cl and methyl.

12. The compound of formula I, a pharmaceutically acceptable salt thereof or stereoisomer thereof according to claim 1, wherein 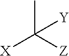
is selected from 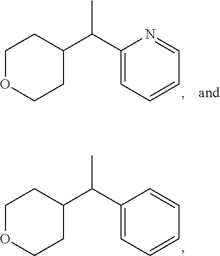
in which pyridine ring is optionally substituted with one substituent, and said substituent is —F.

13. The compound of formula I, a pharmaceutically acceptable salt thereof or stereoisomer thereof according to claim 1, wherein R² is selected from —COOR²¹ and —(CH₂)ₙ—CR²²R²³—OH, wherein R²¹ is (C₁-C₆)alkyl, each of R²² and R²³ is independently —C₁₋₆alkyl; n is selected from 0, 1, 2, 3, 4, 5 and 6.

14. The compound of formula I, a pharmaceutically acceptable salt thereof or stereoisomer thereof according to claim 1, wherein R₂ is selected from —COOR₂₁ and —(CH₂)ₙ—CR₂₂R₂₃—OH, R₂₁ is —CH₃, each of R₂₂ and R₂₃ is —CH₃; n is 0.

15. The compound of formula I, a pharmaceutically acceptable salt thereof or stereoisomer thereof according to claim 1, wherein R¹ is C₁₋₆alkyl; wherein one or more hydrogen atoms on the C₁₋₆ alkyl group are optionally substituted by deuterium.

16. The compound of formula I, a pharmaceutically acceptable salt thereof or stereoisomer thereof according to claim 1, wherein R¹ is —CH₃ or —CD₃.

17. The compound of formula I, a pharmaceutically acceptable salt thereof or stereoisomer thereof according to claim 1, wherein the compound is selected from the exemplified compounds of the description.

18. A pharmaceutical composition which comprises the compound of formula I, a pharmaceutically acceptable salt thereof or stereoisomer thereof according to claim 1 and one or more pharmaceutically acceptable carriers, diluents or excipients.

19. A method of treating diseases or conditions for which a bromodomain inhibitor is indicated in a subject in need thereof, the method comprising administering to the subject a therapeutically effective amount of the compound of formula I, a pharmaceutically acceptable salt thereof or stereoisomer thereof according to claim 1.

20. A method for inhibiting a bromodomain, the method comprising contacting the bromodomain with the compound of formula I, a pharmaceutically acceptable salt thereof or stereoisomer thereof according to claim 1.
